SEQUENCE LISTING (5 sequences)

SEQ ID NO: 1 - protein, 1024 aa, Escherichia coli
  beta-Galactosidase
MTMITDSLAVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSAEFQLSAGRYHYQLVWCQK

SEQ ID NO: 2 - DNA, 2322 bp, Staphylococcus aureus
  mecA-Gen
atgaaaaagataaaaattgttccacttattttaatagttgtagttgtcgggtttggtatatatttttatgcttcaaaagataaagaaattaataatactattgatgcaattgaagataaaaatttcaaacaagtttataaagatagcagttatatttctaaaagcgataatggtgaagtagaaatgactgaacgtccgataaaaatatataatagtttaggcgttaaagatataaacattcaggatcgtaaaataaaaaaagtatctaaaaataaaaaacgagtagatgctcaatataaaattaaaacaaactacggtaacattgatcgcaacgttcaatttaattttgttaaagaagatggtatgtggaagttagattgggatcatagcgtcattattccaggaatgcagaaagaccaaagcatacatattgaaaatttaaaatcagaacgtggtaaaattttagaccgaaacaatgtggaattggccaatacaggaacacatatgagattaggcatcgttccaaagaatgtatctaaaaaagattataaagcaatcgctaaagaactaagtatttctgaagactatatcaacaacaaatggatcaaaattgggtacaagatgataccttcgttccactttaaaaccgttaaaaaaatggatgaatatttaagtgatttcgcaaaaaaatttcatcttacaactaatgaaacagaaagtcgtaactatcctctaggaaaagcgacttcacatctattaggttatgttggtcccattaactctgaagaattaaaacaaaaagaatataaaggctataaagatgatgcagttattggtaaaaagggactcgaaaaactttacgataaaaagctccaacatgaagatggctatcgtgtcacaatcgttagagtcgacgataatagcaatacaatcgcacatacattaatagagaaaaagaaaaaagatggcaaagatattcaactaactattgatgctaaagttcaaaagagtatttataacaacatgaaaaatgattatggctcaggtactgctatccaccctcaaacaggtgaattattagcacttgtaagcacaccttcatatgacgtctatccatttatgtatggcatgagtaacgaagaatataataaattaaccgaagataaaaaagaacctctgctcaacaagttccagattacaacttcaccaggttcaactcaaaaaatattaacagcaatgattgggttaaataacaaaacattagacgataaaacaagttataaaatcgatggtaaaggttggcaaaaagataaatcttggggtggttacaacgttacaagatatgaagtggtaaatggtaatatcgacttaaaacaagcaatagaatcatcagataacattttctttgctagagtagcactcgaattaggcagtaagaaatttgaaaaaggcatgaaaaaactaggtgttggtgaagatataccaagtgattatccattttataatgctcaaatttcaaacaaaaatttagataatgaaatattattagctgattcaggttacggacaaggtgaaatactgattaacccagtacagatcctttcaatctatagcgcattagaaaataatggcaatattaacgcacctcacttattaaaagacacgaaaaacaaagtttggaagaaaaatattatttccaaagaaaatatcaatctattaaatgatggtatgcaacaagtcgtaaataaaacacataaagaagatatttatagatcttatgcaaacttaattggcaaatccggtactgcagaactcaaaatgaaacaaggagaaactggcagacaaattgggtggtttatatcatatgataaagataatccaaacatgatgatggctattaatgttaaagatgtacaagataaaggaatggctagctacaatgccaaaatctcaggtaaagtgtatgatgagctatatgagaacggtaataaaaaatacgatatagatgaataacaaaagcagtgaagcatccgtaacgatggttgcttcactgttttattatgaattattaataagtgctgttacttctccttaaatacatttctcatttcatgtatgttgaaagtgacactgtaacgagtccattttctttttttatggatttcttatttgtaatttcagcgataacgtacaatgtattacctgggtatacaggtttaataaattttaacgttattcatgttgtgttcctgctacaacttcttctccgtatttaccttcttctacccataatttaaatgatattgaaagtgtattgcatgc

SEQ ID NO: 3 - DNA, 14396 bp, Homo sapiens
  SV2A-Gen
ttctccccgtgccctgactccctccccatcctcttttctcctctccccaccaatggtctctcccgatgctcagtggcgcggggtaggggggggcggaggcggggctggagtggtggaaggggggtggcaggtctgcattgccgcttccctggtgccgggagcagtcgccgctgccgcctccgcccgcggccgggacccccgtcctcgcccgggactccttacccggggaacctagaccaggtgagacccctttctcagtcgctcacctcctccccgcccctggcagaagcatctttgccttgccttgcagccattttatcctccgatgctttgcttatggggagggtgacgatcccctctctaggggtgaggggggttggtgggttggtggtggtttggggatggacatttatttcagttgtgctcttcaggacacacacacacacacgcatatgtataatattggtgaatggggaggaggggtgtttttctctaccttaaccaagtccccactatccccttcggagattaacgtggtggtggggttgtgaggtgggatgaggttccctcaaactcctatcccctcctcaccttgacgtccccccacccccaggatgaatgataaatgaccttggctggggctggagaatggatgctgcctcagtgtgcacgcctggacttgggtgggtggggggcaggggaagaagggcaggtgaaggatggagggaaggggagtgaagaatggaaatactggggagacagagatgtagagccgccaggtgttgggggcatggatggcgggggctggcggcagttgctccggggtggggggaagggatgttgagtgaaggtggacacttgtctagcagcaggaccaagggtggaggaggtgctcagagcagggatttagtcagttctgggggcttttcccttattccggagctgaggcaagatggggtgtgggggggtcgctgcaaatgctgataggggcggtctctatgtctgtgcatgcggaggtctctccctgcgtccccctctctgcctctccgtattcctcagtgtgcattctcagctgacaccctcatttttatccctcagaactgtgggtgctcctatcttcctaaccttggtaggatgtggggaggggaggcgcagtcttgatggtcctgggtgggggctgaagttcgatgaccgggctgtcatggggaaggggtgctcccgcacctcccacattctacccccgcccccgcggacgttttgataagtgattgctttttaatgaccttgtcacctgagctaggaaggcctggggatctgggtggaccccaaagtcaggggctttgactgggagggtggggggttctggacccagggctggatgagctgttctgaaagaaagcatttgctctggagtcccctgcctgtctccaccctctgcagaaaatctcctgggcacattttttcctgtcaggtgaagtcagccagcctaggaccttgtaaccctgaaccttgtctcctttaacctccacctctccaagtcttgtctcactcccccaggcttagccaccctgtgctgttttgggggctaaaaatagcaccacttcctcaccagctcaaagaatttgtcttctttctttctctcctgacctcctgggcttgctggccctgaggaagctggctgttgagacctcatatcctgctgtggttgtggaaggcttccctgccctacgtgcccctctcagggggaaatagggtcaacaaacatagagtatggctcctggtgggtcctgcctccttcagggctggaggggagatcctggttaggaaagagaggaaagctagcaaattgccatgcgctgaggggattccgcagttccagctctttcatgggggatggttgggccctgaaccatgaagaaaaggtgacataggggtcactgcttctggtcaccgtggtccactgcttacatctgtcctgaggggataaggcgctgcttgccagtgcttctctctgaaccacatagcattgcttaccgtcttctaccagggcgtgtgtgtgtgtgtgcgtgtgtgtgtgtgtgtgtgtgttttaatgaaggccctggatgggtgggagcaccggaggcaggatatttttagggggatgatttctgatcagaatctaggcctgcttcctctgctcctatgaaggaagggggctatcccatgccttcctctggagaatgtcttttttggggggacacccaaaccccctttattctacatccgcctcctacgcaatgcggtgtttgtggactccttcttccttgactcctggaggaggagcagaaaggattaagggggattaggaagggaggccctggagggatgggatgcaactgtaggccttagaacctcatgttggaaatgtatgcatttgacagtcatttccattctcacccccatctgtggaaagggggcctatctatgacattctgaagcaaggtggcagtggacagtcatcacctctttcttagctttccatctttcctccactttacttgggttccaggtcccccttttctggatcttcccttgttcttctggaggtagttaatgtgggcccagctactggaattagttgaaacatagataggggtgtcttgggactctgtttgcaaggatgctgggcaacagcatctaagtatgacccccctgttgtcccttgctgcttcctgtttctttcccactttcaacatcataggaaggaagaaaactaacatagagtatctactgtgagattacattctcacatcaactccaagaaacagtctcccaatttaattatccccagttaaaatagatggacactgaggcttagggaggttaactaccctgactgagatcagttagtaagtggcagagttgggatttcaacttaggtcacccctaacagtcatgtgctttgccctgaaccaggattctccttgccagtgtcccttcctgctgctcaccctaaagagcatccaagatagctttcatctggggaccttatgaggtagtatagggcatttcactatattactgtcaccacaaaattaataattaatatttattcatcatttttatgtgccaggtgacttatggtaaggagcatagatacattatatcattcgacccttactttcccctttttaaacaagaagaaactgaggcttggtcccttagctagtatttgccaaaacaggaagtcaaccccatgtctttgtgactctggaagctgagcatttacccattagactatgttgagagaaaatccctccttgttgaagcctgtaaacatgggacctgagggaaggacagtggaagcccctgccttcagatgccatcagaggagggaaggccacaggttggagggcttgtcctcttacacatcctcctcaactccacccctagtaagaaacccaggcaagactaaagaagggaattctggtttctcatatttcctgtttagttctttgcagattggtgataaagcaagggctggggtgggtttgggggtaggtgactgcgactttggagcaaattccacaggccaggcctccaaggtcaaagtaaggggacttctatgtgagtgacccggttagcccagatagccatccctttttagtgtgtggatgggagcctaaagcgagaagaccatctctgcaccacatggtccagatggggctctgggttgagaggctttatactggccttcttcaccaccttaggactcacaacttccttttgctgtctgttattttttctaggtctccagaggcttgtggaagagaagcaggcgacccttcctgagttatcctggcttagcctcccaatctggctccccttccccttcccattcccctgctccccctgtcccttccccatccacccaactgaactgggtataggtcaaagctcctctctttccttttccttcctaggcactcattggctaggacctgtttgctcttttttttgtgcccagagatactggaacacgcttcatctaagtaactgtggggaggggtctttttgactctacaagtccttgagcaaaaagctgaaaaagaagcaggaggtggagaagacccagtgaagtgccccaagccccatcatggaagagggcttccgagaccgggcagctttcatccgtggggccaaagacattgctaaggaagtcaaaaagcatgcggccaagaaggtggtgaagggcctggacagagtccaggacgaatattcccgaagatcgtactcccgctttgaggaggaggatgatgatgatgacttccctgctcccagtgatggttattaccgaggagaagggacccaggatgaggaggaaggtggtgcatccagtgatgctactgagggccatgacgaggatgatgagatctatgaaggggaatatcagggcattccccgggcagagtctgggggcaaaggcgagcggatggcagatggggcgcccctggctggagtaagggggggcttgagtgatggggagggtccccctgggggccggggggaggcacaacgacggaaagaacgagaagaactggcccaacagtatgaagccatcctacgggagtgtggccacggccgcttccagtggacactgtattttgtgcttggtctggcgctgatggctgacggtgtggaggtctttgtggtgggcttcgtgctgcccagcgctgagaaagacatgtgcctgtccgactccaacaaaggcatgctaggtaagacatggggggctccagccttatcccctcaaactctggaaacctccttgctcctggaaattgctcttgtccttaccactgggttccctaggaccctgcacccaagatgttcccagagggctcagggttcctctgcacactcttccctcacacactccctcttcttggccttgggtcagggtgaagttgaatgtgacagaggggcttgcgggagggcagctggactggggcttgggccagtggttgggtggtggtctgcttcattctgcaggaggtggctgtggtaggatggtccctaccatttaatgtttcttcatagctttccctctggttatcctgttccctttcactgattggggaacaagaagccagtatccaaactccttggtataaatggggagagtcagtttggggtggttgggggagggggggagggtcctggatcttctgccagagaagctggttagactttctctcactcagatgggagtttccaggggacaggatgtagtctttgatggcctttcctgacagggactctgaggaggaaccccgtagactcacagagaaagaatcgaggccttactctgccaccaaattgccatggggtcttggacaggtctcttcttcctggggcttggtttgggcatgggaaaatgcaaagtttggagtagatgattttaaagattaattccagctttggttttatgaaggcagtgaaaaacaggagagtttttggaccagaaatattgagactggaaggagtaaaggatggattttgtgtgggagatttcctgggaagatgaaatgaattggagcccctttccccaggaacaaatgagagctctcctgctgctttgagggatcaaagcagggagaatagagaggaccctggggtggtctgtgatcctaggcacacccattcctccttcaaagtgagccaggggtaaaggtgtcactgtgtgtgtgtcagagtgagaaatgagacagaccgaagagagtggagagtttgcaggttctcccatatcttcattcctgcctttgaacccctggcctgcagagatgatcttctatctttgcttacaccattcattttctctcacttgctctaatctctgggtagacttaccttctagaagtttttcagatccttcagctccctcagtgctcatctcctgggagaattaaggtgtgtctgctcacaccccctctgcctgttttcctcctccttccaggcctcatcgtctacctgggcatgatggtgggagccttcctctggggaggtctggctgaccggctgggtcggaggcagtgtctgctcatctcgctctcagtcaacagcgtcttcgccttcttctcatcttttgtccagggttacggcactttcctcttctgccgcctactttctggggttgggtgagtgtacacttcctaagtcccttgagggcaggactgtgccttggtcactgttgtatctttagggcccagcacagtgccaggcacaccaaatgcaccttaaatctgtgtgagttgaacggatgaatgagttcctttctcccccagccatcttcaggccagctcctccctgcccaggacctgtctgctgctgtccagaatgctgttccttggcccactcctgagtcctccaccccacctctagttccatttttgagcctgcctccacaaagaggcctagtgggcctcagccccacccctgggctcctgtggacccagagttaccccctaccagagtctacacatttcctgcattacacaacaaaccttgctaacacccacacctgccagaaccctgacgccttgtccgcactgccccttcacttttgcaagcatggcaggcccgcttactctgcttcaactccaagctcctttctggccattcccacaacatctgcccttggaggcttttggcttgtcttttttctctggagatttattctcttttctgcaattactttcccgcccttccaactctgtaattcattttcaagatttaagatttgctctttccctttccctgagactcgcttgcctgtttcccatggtcccaaggatgggtgcttgatggctggcccacatgggagcttgtaaggctttatgtgtgagatggagtctcttcaaaagcctggaagacaaaaggggctcggggtccagagcagtggggcaggggatcaggactatggccatttgctaatgctgaattcttgaaatgcacttcaggattggagggtccatccccattgtcttctcctatttctccgagtttctggcccaggagaaacgaggggagcatttgagctggctctgcatgttttggatgattggtggcgtgtacgcagctgctatggcctgggccatcatcccccactatggtgagcagcccccggaaatccaccccaggttatctccctcctccctcttcttctgtgctccctgtggtgggttcccccagcctctggcctctctgctgatgctgtcactttctccctaccagggtggagttttcagatgggttctgcctaccagttccacagctggagggtcttcgtcctcgtctgcgcctttccttctgtgtttgccattggggctctgaccacgcagcctgagagcccccgtttcttcctagaggtgatggggctgagctgcatggggtggggtggggtgcgggctgtggagtgtggacggcagcaggggcttctgtgttcaaacaccaactggagtttgatttgatcctgtttgactgcctgccttcctcacagagtccactctccaggggactttatcttgcattctttgccttggaatgtgtccctgaggggttcttgggtggagaaaccaggggacctcttcacccatggatgacttaagattttgcagggaagggccttggccctgtcctgcatttacagctcaagagacattcccaccccctataccttcccctcctaaccccagcaggattaggtctttgactacatgcttgcccatcatttcccttacctttggtttttacattcctctgtactatggaattcccaagacctgtgcttgcctttttcccctggacaacactccccacccccaacccgcagctcttgcccaagtctgattgtcttgctatacctcagcttggggctctttcagagtggtgagtgcatgcccataggtcagggcttggtggtggggttgagggctgggaaccatttagtgtcctgtgactacagaagccaggtgggagggagggggtctataactctgggtcaggtaggggctgacttggatgtggggattgtgtctttggctctagaatggaaagcatgatgaggcctggatggtgctgaagcaggtccatgataccaacatgcgagccaaaggacatcctgagcgagtgttctcagtaagccacagccctcccgactcagacctccacgccctgcagccccagcccctacctggcccctctctgagtaccccccacctcagaccacactctaccgtaaggagattttcagggtatcatatccacctcctgcctcccattttgctaatgcaggggaaactgaggcgcctggccgatgttgggaaacacagagtgtgtgactgcccaccccttctcttgaccccaggtaacccacattaagacgattcatcaggaggatgaattgattgagatccagtcggacacagggacctggtaccagcgctggggggtccgggccttgagcctaggggggcaggtaatggtggacggggtggtagatagaagggagtgggggaagtggagaagaaggcaggactgaagtctatgtgtgtgggtgatgggtgagctgaagggaggtgggagggcagaaaagtggggatctatgggtgctagaacttagtatagtcaaggtctgactttgtctggtcccctgtaggtttgggggaattttctctcctgttttggtcccgaatatcggcgcatcactctgatgatgatgggtgtgtggttcaccatgtcattcaggtgaggagatgggtgggctgggtgatgtgtggtggttgtggtggtgtgtgggctgggagagaggtggggagaatggagatgcaggggacaagcgatgagggaaggtgccatgcagaccccctctgatcctgcagccagctcctctacttcgctagttcactcaacaaacttgtactgtaggctgaccaggattcagtttctattctgtgctgggatgaaacagatgtggtccaaggtcctcctggagtttatgttttggttaagaagcaggacgatgaggctcgtaatcccagcactttgggaggacgaggcgggcggatcatgaggttaggagatcgagaccatcctggctacggtgaaaccccgtctctactaacatacaaaaaattagccgggcatggtggtgggcgcctgtagtcccagctactctggaggctgaggcaggagaatggtgtgaatccgggaggcagaggttgcagtgagtcgagatcgcgccactgcactccagcctgggtgacagagcgagactccgtctcaaaagaaaaaaaagaagtgagacgaaacttgctatgaagcagggtgatgagagaagggtgggggaagggcagtgggagcacttggggcttgggctggggtggggacctaagtcctcaaaggtgaatgggagtcagacaggtggagtgggggtggctggggtggagaagagtgtttttggcagagggaaaagcaaatgcttgggaggtaagaaaaagtatagtggctgctgtgcgagaccctgaagtcccccaggagcgcagcaactgttgaagggaggagcagggagaagctggtgaggctggagaggagcaaggtcctaacagaggttgactccatttctcccttgttctgagatgatacacatttccgttttctcctctgcctgattctaatctctgagccttgtctgaagccctgttgtctgtttccattgtccttgactcttcagctactatggcctgaccgtctggtttcctgacatgatccgccatctccaggcagtggactacgcatcccgcaccaaagtgttccccggggagcgcgtagagcatgtaacttttaacttcacgttggagaatcagatccaccgaggcgggcagtacttcaatgacaagtatgtggggacttttaaacttcacttcttccctgttccttctacctcgtctcttttatgagtctaggggttcaagacaagacatttggggggcaaacacttgtggtttcttctcaatgcccagcttcccattacttcgctaaagagtgagtcttggagggggcatgaggatgacgggggagtgtctttcaccatcctggccttacaggttcattgggctgcggctcaagtcagtgtcctttgaggattccctgtttgaagagtgttattttgaggatgtcacatccagcaacacgtttttccgcaactgcacattcatcaacactgtgttctataacactggtaaggcggggtggctggtgggtgtcagaccaaagggttgggtggaccttgatgagaaggaacctttttcattcactatcttagggttctcaagctgagatccctggacccctgtgggaaggtggcagaggggcctgatagctaccaccaataattctgaaaatctaatggatgttggacattttcctgtaatagaatatctgacaaaaacatcaagtttatttgcctttggctgaaattataactcagtgaggtaaaattaatgcacttttgaagaaggtaaaaatcatccaaaatagcattcttgggaaaaagaaaaacaatcaataaaaggggtatttggtgcggatcattgcatgaatccactcctttttaatagtgattctcaaacttcagtatatgcacacgaaatgctggttttgtgagctagactgaaatttcccacctcatactgggcgagctttttccatgttgcaggaaaacaacaatggataggactttttattattttaaatgtttttctcagaaaaatttcctaagtccacggcatcgctactgcttatagtcagctagcacggacagaaagaatgaataagcagctaagactattaatgggagaacttggacaaagtcacactgttggacagatgatggtcattttctgctctaaagatccctagttcccatggtgatgttatttgatgttttgtgattagcactgtgaataattttgagagcccctcctactaactttcatacatgcagagccctgagccataagccgtaacagcctccctctcccatctgctatgactgcctgcttatcttggcctttgcccacagacctgttcgagtacaagtttgtgaacagccgtctgataaacagtacattcctgcacaacaaggagggctgcccgctagacgtgacagggacgggcgaaggtgcctacatggtatactttgtgagcttcctggggacactggcagtgcttcctgggaatatcgtgtctgccctgctcatggacaagatcggcaggctcagaatgcttggtaagggggagaggctgagtcagatctgatagtaggggccaggggctgcgggaggcagtcaacaggcttctcaccttgggctgtggaagagattggtcagagagttcctctctcatgttttctgtttagggggtgctgcaggatggggtggggactggcacagaagcttctctgggggtaaaggaggtaagcagaattgcaagtggttagggcacaggctttgaggttatgggttttgaatctcagctccaccacctgcctctggcctctggcaaattacctctatgagcctggttgtttcctctgcaaaatggaggtgataatagtacctgtgtcatagggttgatgtgctagatcaatgcatgtaaagcatcaattgccttggcccatggctggcattgaataaatgatggttactgatattattaataaacaattataagggaggttgaggaggagtgaaaagccttccttttggataccttggttggataccttcggaacaagtctcatcctgggctatctgaaagccagctgatgggacagggtgggctgtgggtgggtttcactgtggccaggctcatgttgctctctcctctcctggctccagctggctccagcgtgatgtcctgtgtctcctgcttcttcctgtcttttgggaacagtgagtcggccatgatcgctctgctctgcctttttggcggggtcagcattgcatcctggaatgcgctggacgtgttgactgttgaactctacccctcagacaagaggtagttggaatgtgttggaggcagggcagtggattcaggggaggggtggggtgaggaaggagggctgaatgggaagagcatccttaggggtgagaggatatacatttccattttctcctctaaaatctggtagctttggccgggcacggtgactcatgcctgtaatcccagtgctttgggaggccgaggcaggcagatcacctgaggtcaggagtttgagaccagcctggccaacatggtgaaaccctgtctgtactaaaaatacaaaaagttagctgggtgcctgtaatcgcagctactcaggaggctaaggcaggagaattgcttgaacccgggaggtggagctttcagtgagccgagatcgcaccactgcactccagcctgggcaacagagcaagactccgtctcaaaaaaaaaaaaaaaggaaaaaaaaatctggtagcttttgtatctactcctcaaagtcttaactgttaacctcaatttttggtctcctttccctttgtctctcaatttatttacattcctggtgtacatcctttacctctctgatctctccaaccttctctctccctcttggacactgcctgcatcttcctggacttcacttccctgccctccactcttcctttgcctccctcctgtaccttgcgccgtgctgcactgggggactcctgagcaggaccacagcttttggcttcctgaatgccctgtgtaagctggcagctgtgctggggatcagcatcttcacatccttcgtgggaatcaccaaggctgcacccatcctctttgcctcagctgcccttgcccttggcagctctctggccctgaagctgcctgagacccgggggcaggtgctgcagtgaaggggtctctagggctttgggattggcaggcacactgtgagaccaacaactccttccttcccctccctgccctgccatcctgacctccagagccctcactccccactccccgtgtttggtgtcttagctgtgtgtgcgtgtgcgtgtgcatgtgtgtaaaccccgtgggcagggactacagggaaggctccttcatcccagttttgagatgaagctgtactccccatttcccactgcccttgactttgcacaagagaaggctgagccccatccttctccccctgttagagaggggcccttgcttccctgttccaggggttccagaataggcttcctgccttccccatcattccctctgcctaggccctggtgaaaccacaggtatgcaattatgctaggggctggggctctggtgtagaccatggaccaaaagaacttcttagagtctgaagagtgggcctcgggtgccctctcacatctcctgttggatgctgggggagaagcaataaacctcagccctctggcctccactttcctctcaatttgggctgcaaatatgaagcctgaattttatgaaattagctttctgattcttatttattaatagattaagttctgaggcagctccgcaggactgtgtgtgaatgtgtatgtatacttacatatgtgtgtgcatgtgccatggggcggggggtatcactatactgtcctcaaatataagccaagggtaatttcagcggatgcacacacaaccctgcctcccacagttcctcccctaatctggtttctgtgttgagcctgggatggaggagccctaggccagcctgggataagagtcccacagtctagggagatctgagggcatccgacaaggcccatctccttccctcctcaagaagcagaggcctcctctggagtgagaggctccacccactacagcacaggcgggaatagcacagctgccctcccatgctccctacctgtcccctcacagggaggggagcaggggagggaaagaaaccaggcatctggtcaaaccagcagatcaaaaagcacaaagagctggggcagaggcaggaagcaggggccctcctggcagctcctctgagtggggagaggttgggcagtgagtgagggacccctaatgcagggactagaagcctcagtttccccattttacccttccacacaatagcctctgtaggttaggctgccccatcccaccctactctgtgtggctgctttctttggtgccctcccctcaccccactgtagctgtgacgtgttgtagtttttagatgtttgtaaaatgtttaaaaaaatgttaaaaggaaaaaagtgaaaataacaaaaaagaaaatcaaaattcaccttcgtcatgctgcgtccagtgccccaaccctgtggtcactctccccattttgtaacact

SEQ ID NO: 4 - DNA, 14 bp, Artificial Sequence
  Oligonucleotide
aacatcggccaggt

SEQ ID NO: 5 - DNA, 10 bp, Artificial Sequence
  Oligonucleotide
gcctcagccc

(19) United States
(12) Patent Application Publication
(10) Pub. No.: US 2010/0190158 A1
(43) Pub. Date: Jul. 29, 2010

(54) Rapid Test for Detecting DNA Sequences

(76) Inventors: Dieter Peitz, Hamburg (DE); Martin Zentgraf, Hamburg (DE)

Correspondence Address: MARSHALL, GERSTEIN & BORUN LLP, 233 SOUTH WACKER DRIVE, 6300 WILLIS TOWER, CHICAGO, IL, 60606-6357, US

(21) Appl. No.: 12/518,833

(22) PCT Filed: Dec. 12, 2007
(86) PCT No.: PCT/EP07/63750

§ 371 Date: Mar. 2, 2010

(30) Foreign Application Priority Data
Dec. 13, 2006 (EP) 06400039.1

(51) Int. Cl. C12Q 1/68; C07H 15/00

(52) U.S. Cl. 435/6; 536/17.4

(57) ABSTRACT

The present invention relates to an in vitro method for detecting a target DNA sequence in cells using an oligonucleotide which is labelled with a beta-D-galactopyranoside which, on hydrolysis of the glycosidic linkage, forms a water-insoluble dye. The invention relates in particular to an in vitro method for detecting a target DNA sequence from a group of DNA sequences whose members differ from one another in exactly one predetermined nucleotide position, by using an oligonucleotide which is labelled with a beta-D-galactopyranoside which, on hydrolysis of the glycosidic linkage, forms a water-insoluble dye. The invention additionally relates to the use of such an oligonucleotide in DNA hybridization methods. The invention finally relates to kits for carrying out the above methods.

### FIELD OF THE INVENTION

The present invention relates to in vitro methods for the detection of a target DNA sequence in cells using an oligonucleotide labeled with a beta-D-galactopyranoside, which on hydrolysis of the glycosidic bond forms a water-insoluble dye. The invention relates in particular to an in vitro method for the detection of a target DNA sequence from a group of DNA sequences, the members of which differ from one another in exactly one predetermined nucleotide position, using an oligonucleotide labeled with a beta-D-galactopyranoside, which on hydrolysis of the glycosidic bond forms a water-insoluble dye. In addition the invention relates to the use of said oligonucleotide in DNA hybridization methods. Finally the invention relates to kits for carrying out the aforementioned methods.

### BACKGROUND OF THE INVENTION

The detection of particular DNA sequences in biological samples plays a decisive role in the area of molecular biology. For example, in human and veterinary medical practice numerous infections are diagnosed by the detection of pathogen-specific DNA sequences. Furthermore, methods for detecting particular DNA sequences are also used in the identification of genetic variants of particular human genes. These genetic variants can be used as markers for the course of the disease and for the therapeutic response in a number of diseases, e.g. in psychiatric and neurological diseases.

Usually, for detecting DNA sequences in pathogen diagnostics, amplification techniques are used, for example PCR or RT-PCR, in order to increase the quantity of pathogen-specific DNA contained in the sample. In a first step, usually a DNA purification is carried out starting from the respective patient sample (for example blood, serum, tissue, or the like). Depending on the nature of pathogen to be detected, the purified DNA can be obtained from bacterial cells or from cells of the affected tissue of the patient in question (e.g. in the case of infection with viruses whose DNA integrates into the genomic DNA of the infected host cells). The purified DNA can then be used in the respective amplification method, using primers that are specific with respect to their sequence to the pathogen to be detected.

Another possibility for the detection of pathogen-specific DNA sequences is offered by DNA hybridization methods, in which the presence of a defined DNA sequence is detected by hybridization with labeled probes. A classical way of carrying out DNA hybridization methods is Southern blotting. In this method the DNA isolated from the cells or tissues is, after fragmentation with restriction endonucleases, separated by gel electrophoresis, transferred to a membrane (e.g. a nitro-cellulose or nylon membrane), immobilized and hybridized to an appropriately labeled oligonucleotide probe. As an alter-native, the isolated DNA can be put on a membrane directly and then hybridized (dot blot). Southern blotting is often employed for investigating gene rearrangements.

In addition to Southern blotting, in situ hybridization has also become established in pathogen diagnostics. In situ hybridization means the direct detection of nucleic acid sequences in sections and cell preparations using suitable probes. This method is already used in many laboratories for detecting viral DNA (cytomegalovirus, herpesviruses, etc.) in tissue sections and in cells. In addition, the method can be used for the analysis of structural chromosomal changes.

The probes used in DNA hybridization methods can be labeled in various ways, for example with fluorescent molecules or with groups that can be detected immunologically from reaction with an antibody. Hybridization methods normally also require purification of the DNA to be investigated.

The methods for DNA detection described up to now in the prior art have the disadvantage that relatively expensive equipment, for example PCR thermocyclers, fluorescence microscopes or similar are necessary for obtaining information regarding the presence of the DNA sequence to be detected. Furthermore, these methods only provide an evaluation after a considerable delay, which arises from the downstream processes. Moreover, as a rule the methods first require purification of the DNA used, which involves further costs and expenditure of time.

The problem underlying the present invention is therefore to provide methods by which the presence of particular DNA sequences in cells can be detected reliably and reproducibly. It should be possible to carry out the methods without the need for expensive equipment, and the result should if possible be available within a few minutes. According to the invention, the aforementioned problem is solved by the subject matter of the present patent claims.

It was now surprisingly found that oligonucleotides that are labeled with a beta-D-galactopyranoside, which on hydrolysis of the glycosidic bond forms a water-insoluble dye, can be used advantageously in a hybridization assay for the detection of DNA sequences. In an aqueous environment, the dye formed can be discerned with the naked eye only in the region of lipophilic structures, e.g. lipid-containing membranes of lysed cells, whereas in the aqueous reaction solution it does not at first lead to a perceptible color change. Therefore the labeled oligonucleotides according to the invention are suitable in particular for use in a rapid colorimetric test, in which completely or partially lysed prokaryotic and/or eukaryotic cells, on a suitable support, are introduced into a reaction solution containing the labeled oligonucleotides according to the invention as probes. After hybridization of the oligonucleotides to the DNA released from the cells, cleavage of the beta-D-galactopyranoside takes place, and the dye forms in the region of the lysed cells. Cleavage can for example be effected enzymatically by the addition or release of a polypeptide with beta-galactosidase activity.

In such a rapid test it is not necessary to carry out purification of the DNA from the respective cells before hybridization of the labeled oligonucleotides to the target DNA sequence. The cells are instead used directly in the (partially) lysed state in the reaction solution. Surprisingly, it is also not necessary, before carrying out the color reaction, to separate the oligonucleotide probes hybridized to the DNA of the cells from unbound probes that are present in the reaction solution. The probe molecules freely present in the solution only lead to coloration of the reaction solution after a considerable time delay. Thus, according to the invention, a stable and reproducible detection system is provided, which gives information within minutes about the presence of the DNA sequence being sought in the cells under investigation.

### DESCRIPTION OF THE DRAWINGS

FIG. 1 shows the principle of the ligation detection reaction (LDR). A target DNA sequence (1) is incubated with two oligonucleotides (2) and (3) under hybridization conditions. The two oligonucleotides have at least one terminal segment (4) or (5), in which they are sufficiently complementary to the target DNA sequence to make hybridization of the oligonucleotides to the target DNA sequence possible. Ligation of the oligonucleotides and subsequent PCR amplification of the complete sequence, which is made up of the two oligonucleotides, is only possible when there is complete complementarity to the target DNA sequence (1) in the region of the duplex in which the two oligonucleotides are immediately adjacent to one another.

### DESCRIPTION OF THE INVENTION

The invention relates to methods for detecting particular DNA sequences using oligonucleotides that are labeled with a beta-D-galactopyranoside, which on hydrolysis of the glycosidic bond forms a water-insoluble dye. The invention therefore provides an in vitro method for the detection of a target DNA sequence in cells, comprising
(a) providing the cells on a support, wherein at least a portion of the cells is lysed;
(b) contacting the cells provided on the support with at least one oligonucleotide, which is capable of hybridizing to the target DNA sequence, wherein the oligonucleotide is labeled with a beta-D-galactopyranoside, which on hydrolysis of the glycosidic bond forms a water-insoluble dye;
(c) incubating the at least one oligonucleotide under conditions that make hybridization of the at least one oligonucleotide to the target DNA sequence possible;
(d) detecting the hybridization product by hydrolysis of the glycosidic bond of the beta-D-galactopyranoside,
wherein the formation of a dye in the region of the cells present on the support indicates the presence of the target DNA sequence in the cells.

The method according to the invention relates to the detection of a target DNA sequence, i.e. a DNA sequence with at least partially known nucleotide or base sequence. The target DNA sequence can for example be a gene or a part of a gene. The target DNA sequence that is to be detected by the method according to the invention preferably has a length of about 20-20000 nucleotides, wherein a length of about 50, 60, 70, 80, 90, 100, 200, 300, 400, 500, 600, 1000, 5000 or 10000 nucleotides is particularly preferred. Nucleotide means in the present case the building block of DNA, which consists of a phosphoric acid, a pentose and one of the four bases adenine, cytosine, guanine and thymine. The target DNA sequence can be of prokaryotic, eukaryotic or viral origin. The presence, absence, and the concrete composition of the target DNA sequence selected for investigation can for example allow conclusions to be drawn about the presence of a particular disease (e.g. a genetic hereditary disease or an infectious disease), or it can provide clinically relevant parameters that can influence the further course of a therapeutic treatment (e.g. the presence of resistance to particular antibiotics). Using the methods according to the invention it is possible in particular to identify DNA sequences of bacterial and/or viral origin in biological samples, for example in tissue samples from a mammal. Furthermore, by using suitable probes it is also possible to detect a sequence deviation, for example single nucleotide polymorphisms, in a DNA sequence.

In a first step of the method according to the invention, the cells to be investigated, for which it is assumed that they might contain the target DNA sequence to be detected (target cells), are provided on a support, wherein at least a portion of the cells are lysed. This means that the outer envelope of the cell (and in eukaryotic cells the envelope of the nucleus) was disrupted, so that the DNA contained in the cell was released and is therefore accessible for the next steps of the method. Preferably the DNA is in single-stranded form. The DNA continues to adhere to the support for a sufficient length of time and does not dissolve. The cells to be investigated can be of various origins, depending on the nature of the target DNA sequence to be detected. Normally the target cells to be investigated will be in the form of a mixed sample, which also contains other types of cells in addition to the target cells. This does not interfere with the methods of the present invention, provided that it is ensured that a sufficiently high number of target cells is used in the method. Preferably the cells used in the method of the present invention are those obtained from a swab, e.g. a swab from the oral mucosa.

If the target DNA sequence to be detected is a DNA sequence of a pathogenic bacterium, the cells that are applied to the support can be those that were obtained from a sample of a patient potentially infected with the bacterium, preferably from a human patient. If for example a target DNA sequence from bacteria that usually colonize the nose and/or pharynx is to be detected, the cells can be obtained in the form of a mixed sample (i.e. together with eukaryotic cells, for example mucosal cells from the patient) by taking a tissue sample from this region of the patient's body or by means of a swab. The whole mixed sample can be applied to the support directly—it is not necessary to separate the various cells contained in the sample.

An example of a pathogenic bacterium that colonizes the human nose and pharynx and is important in medical practice is Staphylococcus aureus. Certain strains of this Gram-positive microorganism are resistant to numerous antibiotics that are important in clinical therapy. In particular the strains designated as methicillin-resistant Staphylococcus aureus (MRSA) have in recent years developed into a serious problem. MRSA is responsible for a large number of nosocomial infections, which lengthen the time that the patient occupies a bed and so lead to greatly increased costs for hospitals. It is normally spread by patients or nursing staff, who are colonized with MRSA asymptomatically and therefore remain unrecognized as the source of transmission. Timely identification of these persons acting as carriers by methods that are rapid and are easy to operate can ensure early introduction of suitable hygiene measures and prevent the strains spreading to patients.

MRSA strains can be detected for example with pharyngeal, nasal, wound or perineal swabs. The oligonucleotide probe used for detecting the MRSA strains can be derived from one or more MRSA-specific sequences. DNA sequences that can be used specifically for the identification of MRSA strains are well known by a person skilled in the art. According to the invention it is particularly preferred that the oligonucleotide probe is derived from the nucleotide sequence of the mecA gene, which encodes the modified penicillin-binding protein PBP2a (Song et al. (1987), FEES Letters, 221: 167-171). The mecA gene has a size of about 2.4 kb and can have slight sequence variations in different strains. The oligonucleotide probe employed within the scope of the invention is preferably derived from the nucleotide sequence of the mecA gene shown in SEQ ID NO:2. As largely harmless organisms, for example various strains of Staphylococcus epidermidis, may also possess a mecA gene, within the scope of detection of MRSA the presence of another DNA sequence specific to Staphylococcus aureus should be investigated in a parallel assay. This sequence can, for example, be the DNA that codes for certain pathogenicity factors of Staphylococcus aureus, e.g. DNA that codes for protein A, coagulase, clumping factor, exofoliatins A and B, or similar pathogenicity factors. In addition, of course, a sequence specific to S. epidermidis can also be used as negative control.

The mixed sample that includes the cells to be investigated can moreover be material from local infection processes, for example from wounds, abscesses and lesions. Said material can for example be swabs or biopsy specimens. In the case of closed processes, after disinfecting the skin a percutaneous puncture of the abscess can be performed, and the material obtained from the puncture can be investigated for the presence of particular pathogens by the methods according to the invention. Examples of bacterial pathogens that can be detected in wounds include various species of the genus Clostridium, which can cause myonecrosis (gas gangrene). These include for example C. perfringens, C. septicum, C. histolyticum, C. novii and C. fallax. In this case the oligonucleotide probe can, for example, be derived from the nucleotide sequence of the gene that codes for the alpha-toxin of Clostridium perfringens. Numerous other examples of the identification of clinically relevant bacteria will be obvious to a person skilled in the art from the description of the present application.

In the detection of DNA sequences from bacterial pathogens, the sample that contains the cells to be investigated can, for example, also be liquor, urine, joint puncture specimen, sputum, or similar. Depending on the nature and origin of the sample and/or the type of bacterial pathogen to be detected, it may be necessary or advisable to carry out concentration of the cellular material first, before carrying out the method according to the invention. If the biological sample containing the cells to be investigated is a liquid, concentration can for example be carried out by filtration using a filter with a suitable pore size. The cells can then be transferred from the filter directly onto the support.

Owing to the structure of the bacterial cell wall it may be necessary, especially in the case of Gram-positive bacteria, to perform a lysis or at least a partial lysis of the cells before or after applying the cells to the support, so that the DNA specific to the bacterium is released from the cells. Cell lysis can for example be achieved by treatment with alkaline detergents (Sambrook et al. (1989); Publ.; Molecular Cloning, 2nd edition; Cold Spring Harbor Laboratory Press, New York). With certain bacteria, for example bacteria of the genus Staphylococcus, it may prove useful to carry out enzymatic degradation of the bacterial cell wall first. Suitable enzymes that can be used for this purpose include for example lysostaphin and lysozyme. According to the invention it is irrelevant whether the target DNA sequence is present in the respective bacterium as a plasmid or as a constituent of the bacterial chromosome.

According to another embodiment, the DNA sequence to be detected can be a nucleotide sequence of viral origin. In this case the DNA sequence to be detected can be episomal and/or can be integrated in the genome of the infected cell. It will be apparent to a person skilled in the art that in the detection of viral DNA within the scope of the method according to the invention, only cells obtained from regions or body tissues of patients that could be infected with the corresponding virus are to be considered. As viruses normally have cell or tissue specificity, and do not infect just any cells of the body, the type of cells to be investigated depends on the virus whose DNA is to be detected. The viruses can be those that integrate into the genome of the infected cell, or those that are present in the cell episomally. The target DNA sequence can for example derive from a virus that is selected from the group comprising human immunodeficiency virus 1 and 2 (HIV-1 and HIV-2), human T-cell leukaemia virus and 2 (HTLV-1 and HTLV-2), respiratory syncytial virus (RSV), adenovirus, hepatitis B virus (HBV), hepatitis C virus (HCV), Epstein-Barr virus (EBV), human papillomavirus (HPV), varicella zoster virus (VZV), cytomegalovirus (CMV), herpes-simplex virus 1 and 2 (HSV-1 and HSV-2), human herpesvirus 8 (HHV-8, also known as Kaposi sarcoma herpesvirus) and flaviviruses, including yellow fever virus, dengue virus, Japanese encephalitis virus and West Nile virus. The present invention is not, however, limited to the detection of DNA sequences from the aforementioned viruses, but can be applied without any problem to other viruses important in veterinary and/or human medicine. According to one embodiment the target DNA sequence is a sequence of the human papillomavirus, preferably a sequence of human papillomavirus 16 or 18.

Human papillomaviruses can be present episomally and/or integrated in the genome of the infected cell. Infections with papillomaviruses occur in a large number of mammals, for example in humans, sheep, dogs, cats, monkeys, rabbits and cattle. Papillomaviruses normally infect epithelial cells, leading to benign epithelial or fibroepithelial tumors at the site of infection. Papillomaviruses display host specificity and are divided into various groups on this basis. Human papillomaviruses (HPV) are further subdivided on the basis of their DNA sequence homology into more than 70 different types. These types cause different diseases. HPV types 1, 2, 3, 4, 7, 10 and 26-29 cause benign warts. HPV types 5, 8, 9, 12, 14, 15, 17 and 19-25 and 46-50 cause lesions in patients with a weakened immune system. Types 6, 11, 34, 39, 41-44 and 51-55 cause benign acuminate warts on the mucosae of the genital region and of the respiratory tract. HPV types 16 and 18 are of special medical interest, as they cause epithelial dysplasias of the genital mucosa and are associated with a high proportion of the invasive carcinomas of the cervix, vagina, vulva and anal canal. Integration of the DNA of the human papillomavirus is considered to be decisive in the carcinogenesis of cervical cancer. Human papillomaviruses can be detected for example from the DNA sequence of their capsid proteins L1 and L2. Accordingly, the method of the present invention is especially suitable for the detection of DNA sequences of HPV types 16 and/or 18 in tissue samples, for assessing the risk of development of carcinoma.

The supports on which the cells to be investigated are applied have, according to the invention, a surface consisting of one or more adsorbent materials, which can immobilize the cells to be investigated and macromolecules released from them, in particular DNA molecules, for example genomic DNA, on their surface and so prevent the cells and/or the macromolecules detaching from the support when the latter is immersed in a reaction solution. Basically any support material is suitable, on which the cells and the macromolecules released from them, in particular genomic DNA molecules, are immobilized for long enough to permit hybridization of the target DNA sequence and subsequent dye formation. Materials that are suitable for use as support in the proposed method according to the invention include in particular porous materials, for example cellulose or cellulose derivatives.

The support can be of any shape that is suitable for the respective application of the support. If for example it is intended to collect oral mucosal cells, the support can for example be constructed in the form of a small brush-head, which is suitably designed for collecting cells, e.g. from the nose and/or pharynx. Such brushes are known in the prior art and include, among others, the “Omni-Swab” brushes, which are marketed by the company Whatman GmbH (Dassel, Germany). Similar products are also offered by other suppliers (e.g. QIAgen, Baack Laborbedarf or Isohelix Products). It was found, according to the invention, that when swabs are obtained from the oral mucosa using an Omni-Swab brush, no further steps are necessary to bring about the partial lysis of human oral mucosal cells. In fact, just the operation of sampling, i.e. rubbing the brush along the inside surface of the cheek, is enough to provide adherence of a sufficient quantity of cells from the oral mucosa on the brush and at the same time ensure their at least partial maceration. The membranes forming the nucleus are also disrupted, so that the genomic DNA of the cells is released. The shearing forces acting on the genomic DNA of the mucosal cells in this operation are high enough to transform a portion of the DNA to a fragmented, single-stranded state, which makes hybridization to a labeled probe according to the invention possible. It has proved advisable to leave the Omni-Swab brush to dry for 2-3 minutes after application in the oral region, as this results in even stronger adherence of the cellular material and the DNA on the material of the brush. However, it is of course also possible to obtain the cells to be investigated with conventional means, which are not able to immobilize the cells for the subsequent hybridization steps, and then apply the cells to a suitable support. For example, oral mucosal cells can first be obtained by swabbing using conventional cotton buds, and can in a subsequent step be rubbed off onto a support with a cellulose surface.

According to the invention, at least a portion of the cells must be present in lysed form on the support, to permit the oligonucleotide probe used in the next step to come into contact with the DNA released from the cells. The DNA is preferably in single-stranded form. As already mentioned above, various methods of disruption of bacterial cells are known by a person skilled in the art. If the target DNA sequence is a sequence of a mammal, or the sequence of a virus that has infected cells of a mammal, the cells to be investigated, from which the corresponding DNA must be released, are of animal origin. Numerous methods are known by a person skilled in the art that are suitable for the lysis of animal cells, for example human cells. In contrast to bacterial cells, animal cells do not have a resistant cell wall and can therefore be disrupted easily. Being eukaryotic cells, mammalian cells have a nucleus, which must also be disrupted for the genomic DNA of the mammal to be released. As has been seen, in cases when swabs of the oral mucosa are carried out, the mechanical stresses this imposes on the cells is already sufficient to lyse a portion of the cells obtained and release the genomic DNA from the nucleus. In other cases it may be useful, before or after applying the cells to the support, to carry out additional steps that promote or bring about (partial) lysis of the cells. Such measures are well known by a person skilled in the art and comprise for example repeated freezing and thawing of the sample containing the cells, repeated aspiration and discharging of a solution containing the cells using a pipette, addition of detergents and/or other agents that reduce the integrity of the cell wall, and heating of a sample containing the cells. These measures lead not only to disruption of the plasma membrane and of the nucleus, but as a rule also ensure, at the same time, production of single-stranded DNA fragments that can, in the subsequent hybridization reaction, bind to the labeled probes.

The cells on the support are then brought into contact with at least one oligonucleotide that is able to hybridize to the relevant target DNA sequence. The at least one oligonucleotide has, in at least one segment, a sufficient degree of complementarity to enter into a stable interaction with the selected target DNA sequence by complementary base pairing. This process, known as hybridization, is preferably specific, i.e. the oligonucleotide used as probe hybridizes under stringent conditions almost exclusively to the predetermined target DNA sequence and does not bind to a substantial extent to DNA sequences that are not complementary, or are insufficiently complementary, to the oligonucleotide. It is known by a person skilled in the art that in any hybridization assay there is to a certain extent nonspecific binding of the probe to the DNA to be investigated. However, it is possible, without any problem, to select the sequence of the probe and the other conditions of hybridization in such a way that nonspecific binding of the probe is largely suppressed. Stringent conditions, in which specific hybridization takes place almost exclusively, can for example be achieved by using a salt buffer with a salt concentration from 0.1 M to 1.0 M of sodium or potassium ions and a pH value of about 7.0 to 8.3. The buffer can also contain destabilizing agents such as formamide. To achieve stringent conditions, the temperature should be about 3-8° C., preferably 5-6° C. below the melting point of the oligonucleotide. The complementary segment of the oligonucleotide preferably comprises about 5, 6, 7, 8, 9, 10, 11, 12, 13, 14, 15, 16, 17, 18, 19, 20, 22, 24, 26, 28, 30, 40 or more nucleotides. The segment preferably has, over its entire length, complementarity to a corresponding segment in the target DNA sequence. According to a preferred embodiment, the oligonucleotide is completely complementary to a region of the target DNA sequence.

As used here, the term “oligonucleotide” denotes an oligomer or polymer of nucleotide monomers. Preferably the oligonucleotide is in the form of a single-stranded molecule. Oligonucleotides can be produced by chemical synthesis or through PCR methods. Corresponding synthetic methods are described in detail in the prior art and include for example the known phosphoroamidite method.

The term “oligonucleotide” as used here also comprises such oligomers or polymers that contain modified bases, alongside or instead of the naturally occurring bases adenine, guanine, thymine, cytosine and uracil. Said modified bases comprise for example 5-methylcytosine, 5-hydroxymethylcytosine, xanthine, hypoxanthine, 2-aminoadenine, 6-methyladenine, 6-methylguanine, 2-thiouracil, 2-thiothymine, 2-thiocytosine, 5-halouracil, 5-halocytosine, 5-propinyluracil, 5-propinylcytosine, 6-azouracil, 6-azo-cytosine, 6-azothymine, 5-uracil, 4-thiouracil, 8-thioalkyladenine, 8-hydroxyladenines, 8-thioladenine, thioalkylguanine, 8-hydroxylguanine, 8-thiolguanine, 7-methylguanine, 7-methyladenine, 8-azaguanine and 8-azaadenine, 7-deazaguanine, 7-deazaadenine, 3-deazaguanine and/or 3-deaza-adenine.

Preferably the oligonucleotide consists of nucleotide monomers that are joined together by phosphodiester bridges. It is, however, also possible to use oligomers or polymers in which the sugar-phosphate backbone has been replaced with functionally similar structures, for example with a peptide backbone or with a phosphoramide, thiophosphate, methyl-phosphonate or dithiophosphate backbone. Moreover, oligonucleotides that have one or more modified sugar residues are also covered by the invention. For example, one or more hydroxyl groups can be replaced with halogens or aliphatic groups, or they can be functionalized with ethers, amines or similar structures.

According to the invention, the oligonucleotides can be of any length. It is particularly preferred that the oligonucleotides have a length of about 6 to 50 nucleotides, for example a length of 10, 12, 14, 16, 18, 20, 22, 24, 26, 28, 30, 32, 34, 36, 38, 40, 42, 44, 46, 48 or 50 nucleotides. The oligonucleotides used as oligonucleotide probes can also comprise more than 50 nucleotides, for example 60, 70, 80, 90 or 100 nucleotides. The length and composition of the oligonucleotides depends on the composition of the respective target DNA sequence and the conditions of the hybridization assay, e.g. the ionic strength, which is determined by the buffer used, etc. These parameters are adequately known by a person skilled in the art from the literature.

The oligonucleotide used as probe is, according to the invention, labeled with a beta-D-galactopyranoside. As used herein, beta-D-galactopyranoside refers to molecules that consist of a galactose residue and an aglyconic chromogenic residue and are linked via a beta-glycosidic bond. The beta-glycosidic bond can be cleaved by hydrolysis, e.g. by enzymatic hydrolysis. During hydrolysis there is formation of a water-insoluble dye, which settles as a precipitate. Various beta-D-galactopyranosides that on hydrolysis form insoluble dye precipitates are described in the prior art. These beta-D-galactopyranosides can be used within the scope of the methods according to the invention for labeling the oligonucleotide probe.

According to the invention it is particularly preferred that the beta-D-galactopyranoside which is used for labeling the oligonucleotide probe is an indolyl-beta-D-galactopyranoside. Indolyl-beta-D-galactopyranosides are defined herein as compounds in which the galactose residue is linked via a beta-glycosidic bond to an indolyl residue. The indolyl residue forms the chromogenic residue that is responsible for dye formation. These preferred beta-D-galactopyranosides therefore have the following general structure.

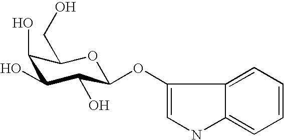

The indolyl residue can be substituted in one or more positions of the ring system. For example, one or more hydrogen atoms of the C—H bonds of the heterocyclic indolyl group can be substituted with a halogen atom, for example a chlorine, bromine or iodine atom. Furthermore, the hydrogen bound to the indole nitrogen can also be substituted, for example with an alkyl group, such as methyl, ethyl or propyl.

According to an particularly preferred embodiment, the indolyl-beta-D-galactopyranoside is 5-bromo-4-chloro-3-indoxyl-beta-D-galactopyranoside, which is supplied under the name x-Gal by various manufacturers (e.g. Fermentas, St. Leon-Rot, Germany; Invitrogen, Karlsruhe, Germany). 5-Bromo-4-chloro-3-indoxyl-beta-D-galactopyranoside is well known from its use in so-called blue-white screening. The colorless x-Gal can be cleaved hydrolytically by the action of a beta-galactosidase to 5-bromo-4-chloro-indoxyl. 5-Bromo-4-chloro-indoxyl is oxidized, in the presence of the oxygen of the air, to a water-insoluble blue dye (5,5′-dibromo-4,4′-dichloro indigo).

Other indolyl-beta-D-galactopyranosides that have a structure comparable to x-Gal comprise for example N-methyl-indolyl-beta-D-galactopyranoside, 5-iodo-3-indolyl-beta-D-galactopyranoside, 5-bromo-6-chloro-3-indolyl-beta-D-galactopyranoside and 6-chloro-3-indoxyl-beta-D-galactopyranoside. These compounds are marketed for example by GENTAUR-BIOXYS, Brussels, Belgium. The galactopyranoside N-methylindolyl-beta-D-galactopyranoside, which is available from GENTAUR-BIOXYS under the name Green-b-D-gal, forms a green dye precipitate on hydrolysis. The galactopyranoside 5-iodo-3-indolyl-beta-D-galactopyranoside, which is available under the name Purple-b-D-gal from GENTAUR-BIOXYS, forms a purple dye precipitate on hydrolysis. The 5-bromo-6-chloro-3-indolyl-beta-D-galactopyranoside marketed under the name Red-b-D-gal by GENTAUR-BIOXYS leads to a red precipitate, whereas 6-chloro-3-indoxyl-beta-D-galactopyranoside available as Rose-b-D-gal produces a pink precipitate.

In addition to indolyl-beta-D-galactopyranosides, other beta-D-galactopyranosides, which on hydrolysis form detectable water-insoluble, nondiffusing complexes, are also known by a person skilled in the art. Such compounds comprise for example cyclohexenoesculetin-beta-D-galactopyranoside and 8-hydroxy-quinoline-beta-D-galactopyranoside, which in the presence of iron ions form black, insoluble complexes. The production of these compounds is described in the prior art (James et al., Appl. and Env. Microbiol. (1996), 62, pages 3868-3870). Another example of a compound that can be considered for use in the method according to the invention is para-naphtholbenzein-beta-D-galactopyranoside, which on hydrolytic cleavage of the glycosidic bond forms an insoluble pink dye (James et al., Appl. and Env. Microbiol. (2000), 66, pages 5521-5523). It is evident that compounds that differ by the replacement of individual groups or substituents from the aforementioned beta-D-galactopyranosides are also covered by the present invention.

The labeling of the oligonucleotide probes with the beta-D-galactopyranoside takes place by coupling of the corresponding beta-D-galactopyranoside to the oligonucleotide. Methods of coupling have been described in the prior art and are within the average ability of a person skilled in the art. According to the invention it is particularly preferred for the coupling to be effected between the phosphate residue of a nucleotide and a hydroxyl group in the sugar ring of the beta-D-galactopyranoside. Coupling is, moreover, undertaken by various commercial suppliers (SEQLAB, Göttingen, Germany; MWG Biotech AG, Ebersberg, Germany; Metabion International AG, Planegg-Martinsried, Germany).

The at least one oligonucleotide is, in step c) of the method according to the invention, incubated under conditions that make hybridization of the at least one oligonucleotide to the target DNA sequence possible. The conditions that affect the hybridization of complementary DNA sequences include, among others, the choice of ionic strength in the reaction batch used, the temperature at which the solution is incubated during the hybridization reaction, and the content of substances that can interfere with the development of the interactions between the complementary DNA sequences (e.g. detergents etc.). A person skilled in the art can match the probe to be used and the reaction conditions to one another without any problem. The corresponding parameters are described in detail in the literature (Sambrook et al. (1989); Publ.; Molecular Cloning, 2nd edition; Cold Spring Harbor Laboratory Press, New York). Incubation can be carried out for a few minutes, e.g. 5-15 minutes, or for up to several hours (e.g. 12-18 h).

It is particularly important to select a suitable temperature during the hybridization of the oligonucleotide probe to the target DNA sequence. This means that the reaction batch should be incubated at a temperature that is high enough to prevent nonspecific binding of the at least one oligonucleotide to the DNA of the cells on the support. However, the temperature should not exceed the melting point of the oligonucleotide, otherwise binding of the oligonucleotide to the predetermined target DNA sequence cannot take place. The melting point Tm of an oligonucleotide is defined (at a defined ionic strength, pH value and DNA concentration) as the temperature at which 50% of the oligonucleotide in equilibrium is hybridized to the target DNA sequence. Calculation of the melting point is known by a person skilled in the art and can be used for establishing the optimum temperature range for the method. It can be estimated approximately from the formula Tm=(A+T)×2° C.+(G+C)×4° C. As used in the present case, the melting point of the oligonucleotides used within the scope of the invention is found from the regions of the oligonucleotides complementary to the target sequence. This means that only the regions in which complementary base pairing with the target sequence takes place are employed for the calculation of the melting point. Since, within the scope of the methods according to the invention, it is also possible to use oligonucleotides that have regions in which there is no complementarity to the target DNA sequence, it is important that only those regions of the oligonucleotides are used for the calculation that hybridize to correspondingly complementary regions from the target sequence. The concentration of monovalent cations (e.g. Na+) and the concentration of double-strand-destabilizing agents (e.g. formamide) are also decisive for the Tm value.

As already mentioned, it was found in the course of the present invention that it is not necessary, after carrying out the hybridization reaction between the oligonucleotide probe and the target DNA sequence, to separate the bound oligonucleotide probes from the unbound oligonucleotide probes. The unbound oligonucleotides only lead to a coloration of the reaction solution after a considerable delay of up to 15 minutes. In contrast, the oligonucleotide bound to the DNA of the cells causes an immediate color reaction. Accordingly, it is possible according to the invention to carry out steps (b) to (d) of the method successively in the same reaction batch, without the need for intermediate steps, e.g. separation of unbound probe.

The detection of the hybridization product formed in step d), i.e. of the DNA duplex of labeled oligonucleotide and target DNA sequence, takes place by hydrolysis of the glycosidic bond of the beta-D-galactopyranoside that is coupled to the oligonucleotide. The hydrolysis is preferably catalyzed by a polypeptide that has beta-galactosidase activity. The polypeptide can be added to the reaction batch after completion of the hybridization step or can be released at this point of time into the reaction batch. Beta-galactosidase activity means herein an enzymatic activity that is able to cleave a beta-glycosidic bond between a galactose residue and an aglycon residue linked to it. Preferably the beta-glycosidic bond is one that involves carbon atom 1 of the galactose residue (for example the beta 1-4-glycosidic bond in lactose). The ability of a polypeptide to cleave the beta-glycosidic bond between a galactose residue and an aglycon residue linked to it can be verified by incubation of the polypeptide under suitable conditions (buffer, pH value, etc.) with a substrate such as x-Gal.

Numerous enzymes with beta-galactosidase activity from prokaryotic and eukaryotic organisms have been described in the prior art. According to a particularly preferred embodiment, the polypeptide with beta-galactosidase activity is the beta-galactosidase from Escherichia coli or a variant or an active fragment thereof. The amino acid sequence of the beta-galactosidase from Escherichia coli is shown in SEQ ID NO:1. Moreover, variants of the polypeptide shown in SEQ ID NO:1 and enzymatically active fragments of the polypeptide and variants thereof are included according to the invention. Variants of a polypeptide are to be understood as those polypeptides that differ by one or more substitutions of amino acids from the amino acid sequence of the polypeptide shown in SEQ ID NO:1. The amino acid substitution can be a conservative or nonconservative amino acid substitution. Basically, any amino acid residue of the amino acid sequence shown in SEQ ID NO:1 can be replaced with another amino acid, provided the substitution does not lead to complete loss of the enzymatic activity. In general, variants of the polypeptide shown in SEQ ID NO:1 will exhibit a significant match with the sequence shown in SEQ ID NO:1. Preferably the amino acid identity is more than 60%, 70%, 80%, 90% or more than 95%. Preferably, the amino acid identity is more than 96%, 97%, 98% or 99%.

Variants further include polypeptides that differ from the polypeptide shown in SEQ ID NO:1 by one or more additional amino acids. These additional amino acids can be located within the sequence shown in SEQ ID NO:1 (insertion) or can be attached to one or both termini of the polypeptide. Insertions can in principle occur at any position of the polypeptide shown in SEQ ID NO:1, provided the substitution does not lead to complete loss of the enzymatic activity of the polypeptide. Variants in which the one or more additional amino acids are attached to the C-terminus and/or N-terminus are particularly preferred. Therefore the term variants also includes fusion polypeptides, in which the polypeptide shown in SEQ ID NO:1 is fused with flanking sequences that permit purification of the protein in heterologous expression. Examples of such sequences include histidine modules, for example 6×His-tag, which through affinity to immobilized nickel ions permit purification of the fusion polypeptide, or domains of protein A, a bacterial cell wall protein from Staphylococcus aureus with a specific activity for the Fc-region of G-class immunoglobulins (IgG). Other flanking sequences that can be used for purification of fusion polypeptides are well known by a person skilled in the art.

Other polypeptides that are also considered to be variants of the polypeptide shown in SEQ ID NO:1 are those in which one or more amino acids are missing compared with the polypeptide shown in SEQ ID NO:1. Such deletions can involve any amino acid position of the sequence of SEQ ID NO:1, provided the substitution does not lead to complete loss of enzymatic activity.

The present invention further comprises enzymatically active fragments of the polypeptide shown in SEQ ID NO:1 and variants thereof as defined above. Within the scope of the present invention, fragments comprise peptides or polypeptides that differ from the polypeptide shown in SEQ ID NO:1 and variants thereof by the lack of one or more amino acids on the N-terminus and/or on the C-terminus of the peptide or polypeptide, wherein at least a portion of the enzymatic activity is retained.

Derivatives of the polypeptide shown in SEQ ID NO:1 or variants thereof are also included according to the invention. Derivatives are, in the present case, polypeptides which, relative to the polypeptide shown in SEQ ID NO:1 or variants thereof, have structural modifications, for example modified amino acids. These modified amino acids can be, according to the invention, amino acids that have been altered either by natural processes, for example processing or post-translational modifications, or by chemical methods of modification that are sufficiently known in the prior art. Typical modifications to which the amino acids of one of the polypeptides according to the invention can be subjected comprise phosphorylation, glycosylation, acetylation, acylation, branching, ADP-ribosylation, crosslinking, formation of disulfide bridges, formylation, hydroxylation, carboxylation, methylation, demethylation, amidation, cyclization and/or covalent or noncovalent binding to phosphatidylinositol, lipids or flavin. Such modifications are widely described in the relevant literature, e.g. in Proteins: Structure and Molecular Properties, T. Creighton, 2nd edition, W. H. Freeman and Company, New York (1993).

It is preferred that the variants or derivatives of the polypeptide shown in SEQ ID NO:1 or the enzymatically active fragments of said polypeptide have up to 75%, preferably up to 80%, 85%, 90%, 95% or even up to 99% of the activity of the polypeptide shown in SEQ ID NO:1.

Apart from the specifically mentioned beta-galactosidase from Escherichia coli, any other prokaryotic and eukaryotic enzymes with beta-galactosidase activity can be used within the scope of the method disclosed according to the invention. Numerous beta-galactosidases that were isolated from prokaryotic organisms are known in the prior art. Examples of bacterial beta-galactosidases comprise for example those isolated from Bacillus cereus, Bacillus halodurans, Bacillus megaterium, Bacillus subtilis, Bacillus circulans, Lactococcus lactis, Lactobacillus acidophilus, Lactobacillus delbrueckii, Streptomyces coelicolor, Bacteroides fragilis, Yersinia pseudotuberculosis, Yersinia pestis, Pseudoalteromonas atlantica, Erwinia carotovora, Thermus thermophilus, Xanthomonas campestris, Alicyclobacillus acidocaldarius, Lactobacillus salivarius, Alteromonas macleodii, Erythrobacter litoralis, Clostridium perfringens, Clostridium beijerincki, Vibrio splendidus, Enterobacter cloacae, Enterococcus faecium, Vibrio vulnificus, Sulfolobus solfataricus, Bifidobacterium adolescentis, Streptococcus pneumoniae, Thermoanaerobacter mathranii, Pyrococcus woesei, Deinococcus geothermalis, Thermotoga maritima, Pyrococcus abyssi, Pyrococcus furiosus, Caldicellulosiruptor saccharolyticus, Streptococcus suis or Xylella fastidiosa.

Moreover, beta-galactosidases of plant origin can also be used, e.g. those isolated from Arabidopsis thaliana, Oryza sativa, Carica papaya, Asparagus officinalis, Capsicum annuum, Mangifera indica, Lycopersicon esculentum, Cicer arietinum, Brassica oleracea, Raphanus sativus, Prunus persica or Sandersonia aurantiaca that have been described with respect to their sequence. Suitable enzymes from fungi comprise the beta-galactosidases from Penicillium canescens, Aspergillus niger, Aspergillus fumigatus, Aspergillus phoenicis, Hypocrea jecorina or Thermomyces lanuginosus.

Furthermore, it is also possible to use beta-galactosidases from animal organisms, in particular those isolated from mammals, for example from tissues of dogs, rats, mice or primates. The beta-galactosidases listed above can be isolated from the relevant organisms or can be expressed recombinantly in other organisms.

Of course, active fragments, variants or derivatives of these beta-galactosidases can also be used, where the terms active fragments, variants and derivatives are to be understood analogously to the definitions given above in relation to SEQ ID NO:1.

The polypeptide used for cleavage of the glycosidic bond can also have, in addition to beta-galactosidase activity, another enzymatic function as well, e.g. in the case of a bifunctional fusion polypeptide (see e.g. Bulow, Eur J Biochem (1987), Vol. 163, pages 443-448).

The polypeptide with beta-galactosidase activity is added to or is released into the reaction batch, after allowing the at least one oligonucleotide sufficient time to hybridize to the target DNA sequence from the cells. This can be achieved for example by pipetting the corresponding enzymatically active polypeptide (optionally in a suitably concentrated buffer) into the reaction batch. According to a particular embodiment of the present invention the enzymatically active polypeptide is added to the reaction batch in the form of wax beads, wherein the wax has a melting point that is above the temperature applied for hybridization. Therefore no release of the enzymatically active polypeptide occurs during the hybridization step. On completion of hybridization of the probes to the DNA target sequence, the temperature of the reaction mixture is increased, so that the wax beads melt and the enzymatically active polypeptides are released. In this step it is necessary to ensure that the temperature that causes the wax beads to melt is not so high that the melting point of the labeled oligonucleotide is exceeded, as otherwise the oligonucleotide would become detached from its target sequence owing to the high temperature.

On completion of hydrolytic cleavage of the glycosidic bond of the beta-D-galactopyranoside, formation and precipitation of the water-insoluble dye occur. The dye is precipitated in the region of lipophilic structures, e.g. in the region of the lysed cells, cell membranes, etc. that are on the support, so that these regions on the support are colored. The coloration can be detected by visual inspection. The coloration indicates the presence of the target DNA sequence in the cells applied to the support.

The methods described above can be used in particular for the identification of an unknown nucleotide at a predetermined position of an otherwise known DNA sequence, for example in the detection of a single nucleotide polymorphism in the chromosomal DNA of humans and of nonhuman mammals. Single nucleotide polymorphisms (SNP) are the commonest sequence variations occurring in mammals. In a single nucleotide polymorphism there is replacement of an individual nucleotide in a particular segment of a DNA molecule (e.g. a gene). In the human genome, a single nucleotide polymorphism is found roughly every 1000 base pairs. If this substitution is located in the coding region of a gene, it can result in an amino acid substitution in the encoded polypeptide. Therefore the substitution of a single nucleotide can have serious physiological consequences for the individual in question. Single nucleotide polymorphisms have been described in connection with a number of diseases, for example in Alzheimer's disease, cystic fibrosis, mucoviscidosis, Duchenne's muscular dystrophy, Huntington's chorea, sickle cell anemia and malignant hyperthermia. Furthermore, single nucleotide polymorphisms play an important role in allergic drug reaction (e.g. to carbamazepine) and in Stevens-Johnson syndrome (toxic epidermal necrolysis). Moreover, responsiveness to ovarian stimulation in reproductive medicine and to beta-adrenergic drugs in chronic obstructive lung disease is affected by single nucleotide polymorphisms.

A number of different methods of detecting single nucleotide polymorphisms are known in the prior art. As a rule methods are applied that are based on amplification of the region of the sequence of the patient's genomic DNA that contains the single nucleotide polymorphism. An elegant possibility for detecting a single nucleotide polymorphism was described by Langegren and Hood in U.S. Pat. No. 4,988,617. This method has become known in the field as ligation detection reaction (LDR). The method is based on the hybridization of two oligonucleotides to a target DNA sequence, wherein the two oligonucleotides hybridize immediately next to each other to the target DNA sequence and in a subsequent step are joined together by a ligase. The longer oligonucleotide formed as a result of ligation is detected by PCR, wherein formation of a PCR product only occurs if ligation of the two individual oligonucleotides has taken place. Such ligation in turn only occurs if there is complete base pairing with the target DNA sequence in the adjacent end regions of the oligonucleotides. If, however, there has been no base pairing with the target DNA sequence in this region of one of the nucleotides (for example owing to a single nucleotide polymorphism in the target DNA sequence), no ligation of the oligonucleotides occurs, and therefore no PCR product can be amplified. Based on this principle, by using the labeled oligonucleotides according to the invention, it is possible to devise a rapid test that can provide information about the presence of the respective single nucleotide polymorphism within a few minutes, without having to resort to PCR or other expensive methods of detection.

According to a particular embodiment of the present invention, an in vitro method is therefore provided for the detection of a target DNA sequence from a group of DNA sequences, the members of which differ from one another in exactly one predetermined nucleotide position. The method comprises
(a) providing cells on a support, wherein at least a portion of the cells is lysed;
(b) contacting the cells on the support with a first and a second oligonucleotide, at least one of which is labeled with a beta-D-galactopyranoside, which on hydrolysis of the glycosidic bond forms a water-insoluble dye, and wherein said oligonucleotides are selected so that
   (i) in each case they can hybridize by a terminal segment to the target DNA sequence, in such a way that the terminal nucleotides of the two terminal segments are arranged immediately adjacent to one another, wherein the nucleotide at the predetermined position of the target DNA sequence forms a complementary base pairing with a nucleotide in the terminal segment of the first oligonucleotide, and so that
   (ii) on hybridization of the two terminal segments to another DNA sequence from the group of DNA sequences due to the absence of complementary base pairing between the nucleotide at the predetermined position of the DNA sequence and a nucleotide in the terminal segment of the first oligonucleotide, the terminal nucleotide of the terminal segment of the first oligonucleotide cannot hybridize to the DNA sequence, so that the terminal nucleotides of the two terminal segments are not arranged immediately adjacent to one another;
(c) incubating the two oligonucleotides under conditions that make hybridization of the two oligonucleotides to the target DNA sequence possible;
(d) contacting the two hybridized oligonucleotides with a ligating agent which only links them to form a ligation product if the two terminal nucleotides of the terminal segments are arranged immediately adjacent to one another;
(e) heating the reaction batch to a temperature at which labeled, nonligated oligonucleotides detach from the target DNA sequence, and at which the ligation product remains hybridized to the target DNA sequence;
(f) detecing the ligation product hybridized to the target DNA sequence by hydrolysis of the glycosidic bond of the beta-D-galactopyranoside;
wherein the formation of a dye in the region of the cells on the support indicates the presence of the target DNA sequence in the cells.

The target DNA sequence to be detected is preferably the sequence of a gene or of a part of a gene. According to an particularly preferred embodiment it is the sequence of a human gene or of a part thereof. According to the invention the target DNA sequence is known completely or partially. This means that at least the region around the predetermined nucleotide position is known with respect to its nucleotide or base sequence, so that corresponding oligonucleotides can be prepared for hybridization to this region. The method serves for distinguishing the target DNA sequence from other DNA sequences that differ in at least one, preferably in exactly one predetermined nucleotide position (i.e. by one base) from the target DNA sequence. The term “predetermined” means in this context that the position at which the various DNA sequences differ from one another is known with respect to its localization in the DNA sequences. The target DNA sequence preferably has a length of about 20-20000 nucleotides, where a length of about 50, 60, 70, 80, 90, 100, 200, 300, 400, 500, 600, 1000, 5000 or 10000 nucleotides is particularly preferred.

The members of the group of DNA sequences can for example be various allelic variants of a human gene. If for example the sequence of a particular human gene is examined, which in the relevant coding sequence has a single variable position at a known position, then theoretically in addition to the wild-type sequence, three further sequence variants are conceivable, each having a nucleotide with another base at this position. This means that the target DNA sequence and the three variant sequences form a group, the members of which each differ exactly at one predetermined nucleotide position from the other members of the group.

Again, cells that are provided on a support, wherein at least a portion of the cells is lysed, so that the DNA has been released from the cells and adheres to the support, serve as the starting point of the method. Preferably the DNA is in single-stranded form. The cells are preferably obtained from a mammal, for example a human, and it does not matter which cells of the body are used for the method. As it should be possible to detect the nucleotide to be identified (e.g. a single nucleotide polymorphism) in the genomic DNA of every cell of the mammal's body, cells that are particularly easy to lyse and/or can be obtained particularly easily can be selected for the method. The cells can be mucosal cells, preferably human mucosal cells. Human oral mucosal cells are particularly preferred. The materials described above can be considered as the support, and the use of “Omni-Swab” brushes or comparable products is particularly preferred.

The cells provided on the support are contacted with a first and a second oligonucleotide, wherein at least one is labeled with a beta-D-galactopyranoside, which on hydrolysis of the glycosidic bond forms a water-insoluble dye. It is equally possible, however, to label both oligonucleotides with the same beta-D-galactopyranoside or with different beta-D-galactopyranosides. This leads, because of the higher number of markers, to intensification of the signal. The beta-D-galactopyranosides that can be used in this method were described above. According to a preferred embodiment the beta-D-galactopyranoside is selected from the group comprising 5-bromo-4-chloro-3-indoxyl-beta-D-galactopyranoside (x-Gal), N-methylindolyl-beta-D-galactopyranoside, 5-iodo-3-indolyl-beta-D-galactopyranoside, 5-bromo-6-chloro-3-indolyl-beta-D-galactopyranoside, 6-chloro-3-indoxyl-beta-D-galactopyranoside, para-naphtholbenzein-beta-D-galactopyranoside, cyclohexenoesculetin-beta-D-galactopyranoside and 8-hydroxy-quinoline-beta-D-galactopyranoside. According to an especially preferred embodiment the beta-D-galactopyranoside is 5-bromo-4-chloro-3-indoxyl-beta-D-galactopyranoside (x-Gal).

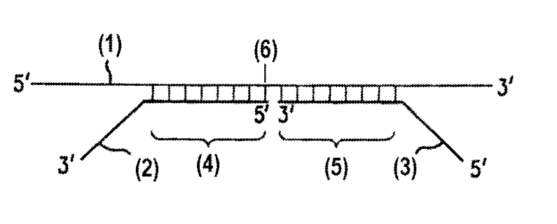

The oligonucleotides are selected so that in each case they can hybridize with a terminal segment to the target DNA sequence. This means that each of the two oligonucleotides has at least one terminal segment that is sufficiently complementary to a corresponding segment of the target DNA sequence to make hybridization to the target DNA sequence possible. Preferably the segment allows a specific hybridization, i.e. hybridization only to the intended segment of the target DNA sequence and not to other sequences that have little or no similarity with the target DNA sequence. The segment complementary to the target DNA sequence is terminal, i.e. it comprises a terminus (either the 5′-terminus or the 3′-terminus) of the respective oligonucleotide and extends in the direction of the opposite terminus of the same oligonucleotide. It is preferred that the terminal segment of the oligonucleotides necessary for hybridization to the target sequence has a length of at least 5, 6, 7, 8, 9, 10, 11, 12, 13, 14, 15, 16, 17, 18, 19, 20, 22, 24, 26, 28, 30, 40, 50 or more nucleotides. It is not absolutely necessary for every nucleotide in this segment to be complementary to the corresponding nucleotide in the target DNA sequence, provided the segment is on the whole sufficiently complementary to be able to hybridize with a sufficiently high specificity to the predetermined segment of the target DNA sequence. It is preferred that the terminal segment of the oligonucleotides has a complete complementarity to a corresponding region of the target DNA sequence. Of course, oligonucleotides that are completely complementary to the target DNA sequence can also be used according to the invention. According to a particularly preferred embodiment, therefore, the first and/or second oligonucleotide is complementary over its entire length to the target DNA sequence. If the oligonucleotides have segments that have no complementarity to the target sequence (e.g. as shown in FIG. 1), these segments can be used for labeling with the beta-D-galactopyranoside. Labeling can, however, also take place at any other positions of the oligonucleotides.

The terminal segments of the first and second oligonucleotide hybridize immediately next to one another to the target DNA sequence, i.e. after hybridization of the terminal segments to the target DNA sequence, the two terminal nucleotides of the terminal segments are arranged immediately adjacent to one another. The terminal nucleotides of the terminal segments are arranged immediately adjacent to one another if they hybridize to two immediately adjacent nucleotides in the target DNA sequence. Hybridization of the two terminal nucleotides of the terminal segments to respective complementary nucleotides in the target DNA sequence is a necessary precondition for the subsequent linkage of the two oligonucleotides to form a ligation product. It is only in this immediately adjacent arrangement that the two terminal nucleotides of the contiguous terminal segments of the oligonucleotides are close enough together to be joined by a ligating agent, e.g. a ligase. Should one of the two terminal nucleotides (or both) not be paired with a corresponding nucleotide in the DNA sequence to be investigated, ligation of the terminal nucleotides does not take place.

The oligonucleotides are moreover arranged so that the nucleotide at the predetermined position in the target DNA sequence is, after hybridization of the first oligonucleotide, assigned to a nucleotide in the terminal segment of the first oligonucleotide. It is only on hybridization to the required target sequence that complementary base pairing occurs between this nucleotide of the first oligonucleotide and the predetermined position in the target DNA sequence. On binding of the first oligonucleotide to another DNA sequence, which differs from the target DNA sequence in the predetermined position, complementary base pairing does not occur in this position. This mispairing means that the terminal nucleotide of the terminal segment cannot hybridize to the DNA sequence. Consequently, after hybridization to the DNA sequence, the two terminal segments of the first and second oligonucleotide are not in an immediately adjacent position relative to one another, and ligation cannot take place.

For the mispairing at the predetermined position in the case of a DNA sequence that differs from the target DNA sequence to be able to effectively prevent the hybridization of the terminal nucleotide of the terminal segment of the first oligonucleotide, the predetermined position in the DNA sequence to be investigated must be assigned to a nucleotide in the first oligonucleotide that is located near the terminal nucleotide of the terminal segment. Preferably the nucleotide in the terminal segment of the first oligonucleotide that is assigned to the predetermined position in the DNA sequence to be investigated is the terminal nucleotide of the terminal segment (cf. FIG. 1). However, it is moreover also possible for the predetermined position in the DNA sequence to be investigated to be assigned to a nucleotide in the first oligonucleotide that is located one, two or three positions away from the terminal nucleotide of the terminal segment, i.e. the predetermined position of the DNA sequence to be investigated can be assigned to one of the last four nucleotides in the terminal segment of the first oligonucleotide. A mispairing at these positions also has the result that the terminal nucleotide in the terminal segment of the first oligonucleotide can no longer hybridize to an otherwise complementary DNA sequence.

The principle of the binding of the two oligonucleotides is illustrated in FIG. 1 for an example of an embodiment. A target sequence (1) has a predetermined nucleotide position (6). This nucleotide position can for example be the position of a single nucleotide polymorphism. The oligonucleotides (2) and (3) hybridizing to the target DNA sequence (1) are matched to one another with respect to their sequence so that oligonucleotide (2) has a terminal sequence segment (4) that hybridizes upstream (i.e. relative to a target DNA sequence extending from 5 to 3′: in the 5′ direction from the predetermined nucleotide position (6) in the target DNA sequence) to the target DNA sequence. The second oligonucleotide (3) has a terminal sequence segment (5) that hybridizes downstream (i.e. relative to a target DNA sequence extending from 5′ to 3′: in the 3′ direction from the predetermined nucleotide position (6) in the target DNA sequence) to the target DNA sequence. Since the two terminal sequence segments (4) and (5) hybridize completely to the target DNA sequence in the region of the termini, the two terminal nucleotides of both terminal segments (4) and (5) are arranged immediately adjacent to one another, i.e. relative to a target DNA sequence extending from 5′ to 3′, the 5′-terminal nucleotide of the oligonucleotide hybridizing upstream of the predetermined nucleotide position (6) is contiguous with the 3′-terminal nucleotide of the oligonucleotide hybridizing downstream of the predetermined nucleotide position (6).

FIG. 1 shows an embodiment of the method according to the invention, in which the predetermined nucleotide position (6) in the target DNA sequence is positioned opposite to the 5′-terminal nucleotide of oligonucleotide (2). In another embodiment the predetermined nucleotide position (6) is positioned opposite to the 3′-terminal nucleotide of oligonucleotide (3). Furthermore, embodiments are possible in which the predetermined nucleotide position (6) is positioned opposite to the penultimate, third from last or fourth from last nucleotide in one of the terminal segments (4) and (5). In the case shown, complementary base pairing takes place between the predetermined nucleotide position (6) in the target DNA sequence and the 5′-terminal nucleotide of oligonucleotide (2), so that the two terminal nucleotides of both terminal segments (4) and (5) are arranged immediately adjacent to one another. Thus, as the process continues, there can be formation of a ligation product from the two oligonucleotides and therefore dye formation during detection of the ligation product.

In a next step of the method, the two oligonucleotides have an opportunity to hybridize to the target DNA sequence. The conditions permitting hybridization largely depend on the composition of the oligonucleotides. A person skilled in the art will be able to select these conditions without any problem for each individual case. A specific hybridization will in particular occur at temperatures that are high enough to suppress nonspecific hybridization of the two oligonucleotides to noncomplementary regions of the genomic DNA. However, the temperature selected must not be so high that, despite sufficiently complementary segments, the oligonucleotides are unable to bind to the target DNA sequence. Preferably the temperature in the hybridization step of the method is selected so that it is about 5-10° C. below the lower of the two melting points of the oligonucleotides. A person skilled in the art will have no problem in calculating the melting point of the oligonucleotides, wherein only the regions complementary to the target sequence are included in the calculation. The two oligonucleotides are preferably selected so that they both have a similar melting point. This makes substantially uniform hybridization to the target DNA sequence possible.

In a subsequent step the hybridized oligonucleotides are contacted with a ligating agent, which only links them to form a ligation product if the two terminal nucleotides of the terminal segments are arranged immediately adjacent to one another. This means that it is only in the case of a complementary base pairing that ligation can take place between the nucleotide at the predetermined position in the target DNA sequence (e.g. the position establishing the polymorphism) and the corresponding nucleotide in the terminal segment of the hybridized oligonucleotide, as otherwise the two terminal nucleotides of the oligonucleotides are not arranged closely enough together for linkage of the 5′-terminal nucleotide of the upstream hybridized oligonucleotide to the 3′-terminal nucleotide of the downstream hybridized oligonucleotide to be possible. If the nucleotide does not form base pairing at the predetermined position in the DNA sequence being investigated because of insufficient complementarity to the corresponding nucleotide in the terminal segment of the hybridized oligonucleotide, ligation of both terminal nucleotides of both oligonucleotides is not possible, as the 5′-terminal phosphate group of one oligonucleotide is not close enough to the 3′-terminal OH group of the other oligonucleotide to be linked by the ligating agent. Such linkage is only possible if the two terminal nucleotides of the oligonucleotides are held in a correspondingly sterically favorable position by complementary base pairing with nucleotides of the target DNA sequence.

The ligating agent is preferably a DNA ligase. In the present case, DNA ligases are understood to be enzymes that link DNA strands together. They form a phosphodiester bond between a 5′-phosphate residue of one DNA strand and an OH group of the other DNA strand. The DNA ligase can be a ligase from bacteriophage T4. This ligase uses ATP as cofactor. In addition, ligases from thermophilic or hyperthermophilic bacteria can also find application. The use of DNA ligase from E. coli is also possible within the scope of the present invention. This enzyme uses NAD as cofactor. According to an especially preferred embodiment the DNA ligase is T4-DNA ligase. According to another preferred embodiment the ligase is a thermostable ligase, as was described for example by Barany, Proc. Nat. Acad. Sci. USA 88: 189-193 (1991), in connection with such a method.

The reaction batch is then heated to a temperature at which labeled, nonligated oligonucleotides detach from the target DNA sequence and/or from the other DNA sequences of the group, and at which the ligation product that forms remains hybridized to the target DNA sequence. This means that the temperature selected for this step is above the melting point of the labeled oligonucleotides. If unlabeled oligonucleotides are also used, these can also remain hybridized to the target DNA sequence, as they have no effect on the subsequent color reaction. Preferably the selected temperature is above the melting points of both oligonucleotides. If ligation of the two oligonucleotides to form a longer ligation product has not taken place, in this case the two individual oligonucleotides will detach from the target DNA sequence when the reaction batch is heated. If, however, a ligation product has formed from the two oligonucleotides as a result of ligation, this has a longer region complementary to the target DNA sequence and accordingly a far higher melting point. It is therefore possible to set the temperature in this reaction step so that the melting point of the two individual oligonucleotides is indeed exceeded, but not the melting point of the ligation product. The ligation product therefore remains hybridized to the target DNA sequence and can subsequently bring about formation of the dye in the region of the cells applied to the support.

In a last step, the hybridization product is detected by hydrolysis of the glycosidic bond of the beta-D-galactopyranoside. As already described, this color reaction can be achieved through addition or release of a polypeptide with beta-D-galactosidase activity. Preferably the polypeptide is the beta-galactosidase from Escherichia coli according to SEQ ID NO:1 or a variant or an active fragment thereof. According to an especially preferred embodiment of the present invention the release of the enzymatically active polypeptide can be achieved by incorporating the polypeptides, which are added to the reaction batch, in wax beads. The methods for the production and use of these wax beads are well known in the prior art, in particular in connection with so-called Hotstart-PCR methods. Suitable wax beads that are suitable for the incorporation of enzymatically active proteins or polypeptides are described for example in U.S. Pat. No. 5,413,924. As a color reaction within the scope of the above method should only be caused by the hybridized ligation product, the temperature required for melting the wax beads must be selected so that melting only occurs in a temperature range that is above the melting points of both oligonucleotides. In this way, a color reaction is not initiated until the labeled, unligated oligonucleotides have already detached from the target DNA sequence. The temperature for melting the wax beads is further selected so that the ligation product does not detach from the target DNA sequence, i.e. the temperature is below the melting point of the ligation product.

The formation of a dye in the region of the cells on the support indicates directly the presence of the ligation product from the two oligonucleotides. As the formation of the ligation product requires complementary base pairing at the predetermined nucleotide position in the target DNA sequence and the nucleotide of the first oligonucleotide opposite to this position, from the dye formation in the region of the cells on the support it can be concluded directly that the predetermined nucleotide position in the target DNA sequence is complementary to the nucleotide in the first oligonucleotide that is opposite to this. If no dye formation is observed, the method can be repeated with a DNA sequence that has a nucleotide with another base at the predetermined position, until dye formation is observed.

According to a preferred embodiment of the invention, the target DNA sequence is included in the sequence of the wild type of a gene. According to another preferred embodiment of the invention, the target DNA sequence is included in the sequence of the variant of a gene, i.e. in a sequence that differs by a point mutation from the sequence of the wild type of the gene. Preferably the target DNA sequence corresponds to the wild-type or variant sequence of a gene. Thus, for example, the target DNA sequence can be included in the gene that codes for human synaptic vesicle protein 2A. The wild-type gene coding for synaptic vesicle protein 2A (SV2A) has the sequence shown in SEQ ID NO:3. As an alternative, the target DNA sequence can be included in the variant of the gene shown in SEQ ID NO:3 that differs in position 8348 from the wild type.

It was found that the SV2A gene has a single nucleotide polymorphism in intron 6, which is decisive for a patient's responsiveness to particular therapeutic agents. The polymorphism was described in detail in the international patent application (PCT/EP2006/066832) filed on 28 Sep. 2006. The polymorphism relates to the substitution of guanine (wild-type) in position 8348 of SEQ ID NO:3 with an adenine (variant). This single nucleotide polymorphism is a predictor for a patient's response to antiepileptic medication with various substances, for example levetiracetam (Keppra®; available from UCB GmbH, Kerpen, Germany). The substance levetiracetam is described in EP 0 162 036. It is an ethyl analog of piracetam.

Patients who have the base “A” instead of the base “G” in position 8348 of SEQ ID NO:3 have a roughly 5-times greater risk of not responding to a clinically significant degree to said substance. Accordingly, determination of the genotype of an epileptic patient has high predictive value with respect to treatment with levetiracetam. The methods of the present invention make it possible, at the very first encounter between doctor and patient, to determine whether a patient has a high probability of responding to treatment with levetiracetam. This polymorphism can be detected quickly and reliably by means of the methods described within the scope of the invention, wherein for example the oligonucleotides described in SEQ ID NO:4 and SEQ ID NO:5 can be used. The oligonucleotide shown in SEQ ID NO:4 corresponds to the first oligonucleotide of the method according to the invention, which hybridizes to the sequence of the SV2A gene so that the 3′-terminal “T” is directly opposite to the position of the possible polymorphism in position 8348 of SEQ ID NO:3. The oligonucleotide shown in SEQ ID NO:4 therefore hybridizes to a region of the sequence of SEQ ID NO:3 that extends from position 8349 to position 8361. The oligonucleotide shown in SEQ ID NO:5 corresponds to the second oligonucleotide, which hybridizes in the 5′-direction of position 8348 of SEQ ID NO:3. It hybridizes to a region of the sequence of SEQ ID NO:3 that extends from position 8338 to position 8347, so that the 5′-terminal “G” is directly contiguous with the terminal “T” of the oligonucleotide of SEQ ID NO:4. Of course, it is also possible to use longer oligonucleotides, which comprise the sequences of SEQ ID NO:4 and SEQ ID NO:5.

As is apparent to a person skilled in the art, the method according to the invention, which is applied within the scope of detection of a polymorphism, is designed in such a way that the formation of a ligation product (and therefore dye formation in the region of the cells) only occurs when the DNA of the individual being investigated does not have a polymorphism, but corresponds to the wild type. In this case the DNA sequence of the wild type corresponds to the target DNA sequence to be detected, and the nucleotide of the respective oligonucleotide, which is opposite to the position of the single nucleotide polymorphism, is selected so that it has a base that is complementary to the base of the nucleotide that is present at this position in the wild type.

Of course, the oligonucleotides can also be arranged so that when carrying out the method ligation of the hybridized oligonucleotides and formation of the dye only occur when the genomic DNA investigated within the scope of the method according to the invention has a sequence that deviates from the wild type, for example the sequence of a single nucleotide polymorphism. In this case the DNA sequence of the variant or mutant corresponds to the target DNA sequence to be detected, and the nucleotide of the respective oligonucleotide that is opposite to the position of the single nucleotide polymorphism will have a base that is complementary to the base that was described in the course of the polymorphism at this position.

For the reasons already mentioned, it is possible according to the invention for steps (b) to (f) of the method to be carried out one after another in the same reaction batch, without intermediate steps.

According to another aspect, the invention relates to the use of an oligonucleotide that has been labeled with a beta-D-galactopyranoside as defined above, in DNA hybridization methods. In these methods, the oligonucleotide is used as a probe. The beta-D-galactopyranoside is preferably the galactopyranoside 5-bromo-4-chloro-3-indoxyl-beta-D-galactopyranoside (x-Gal).

Furthermore, the present invention provides kits for carrying out the methods described within the scope of the invention. The kits comprise at least one oligonucleotide labeled with a beta-D-galactopyranoside, which on hydrolysis of the glycosidic bond forms a water-insoluble dye. The kits can also comprise one or more other oligonucleotides labeled in this way. The beta-D-galactopyranoside is preferably the galactopyranoside 5-bromo-4-chloro-3-indoxyl-beta-D-galactopyranoside (x-Gal). In addition the kits contain buffers and other reagents that can be used for carrying out hybridization methods. The kits can for example contain a beta-galactosidase or suitable supports, such as the small brushes described above. If the kit serves the detection of a target DNA sequence from a group of DNA sequences whose members differ from one another in exactly one predetermined nucleotide position (e.g. the detection of a single nucleotide polymorphism), it can in addition contain a ligating agent, for example a T4-DNA ligase, and oligonucleotides that hybridize immediately next to one another to a particular target sequence. The kits can, furthermore, contain instructions for carrying out the methods of detection described above.

### EXAMPLES

The following example explains the detection of the single nucleotide polymorphism that relates to synaptic vesicle protein 2A (SV2A). The following reaction solution was prepared in a 1.5-ml reaction tube:

| 40 | U | beta-galactosidase (incorporated in wax beads) |
|---|---|---|
| 20 | U | T4-DNA ligase |
| 50 | μM | ATP |
| 66 | mM | Tris-HCl (pH 7.6) |
| 150 | nM | NaCl |
| 10% | | PEG |
| 6.6 | mM | MgCl₂ |
| 10 | mM | DTT |
| to 1000 | μl | A. dest |

The following oligonucleotides were added:

| | |
|---|---|
| oligo 1: | 5′-AACATCGGCCAGGT-3′ |
| oligo 2: | 5′-GCCTCAGCC-3′-X-Gal |

The 3′-terminal nucleotide of oligo 1 is a “T”, so that base pairing with the genomic DNA of the test subject only takes place at this nucleotide if there is an “A” (instead of a “G”) at the corresponding position in the genomic DNA (position 8348 of SEQ ID NO:3), deviating from the sequence of the wild type. Oral mucosal cells were obtained from a patient by means of an “Omni-Swab” brush (Whatman). After drying the brush in air for 2-3 minutes, the brush-head was put in the aforementioned reaction vessel. The reaction solution was first held at a temperature of 22° C. After 3 minutes the temperature of the reaction batch was increased by means of a thermocouple to 32° C. It was found that in the conditions described above, dye formation on the cellular material of the brush-head in the reaction solution only occurs in test subjects with a single nucleotide polymorphism in the SV2A gene confirmed by sequencing. The formation of the blue dye is already clearly discernible after 5 minutes in the region of the brush. After about 15 minutes there is blue coloration of the entire reaction solution.

1. An in vitro method for the detection of a target DNA sequence in cells, comprising
(a) providing the cells on a support, wherein at least a portion of the cell is lysed;
(b) contacting the cells on the support with at least one oligonucleotide, which is capable of hybridizing to the target DNA sequence, wherein the oligonucleotide is labeled with a beta-D-galactopyranoside, which on hydrolysis of the glycosidic bond forms a water-insoluble dye;
(c) incubating the at least one oligonucleotide under conditions that make hybridization of the at least one oligonucleotide to the DNA target sequence possible;
(d) detecting the hybridization product by hydrolysis of the glycosidic bond of the beta-D-galactopyranoside,
wherein the formation of a dye in the region of the cells on the support indicates the presence of the target DNA sequence in the cells.

2. The method of claim 1, wherein the beta-D-galactopyranoside is selected from the group consisting of from 5-bromo-4-chloro-3-indoxyl-beta-D-galactopyranosid (x-Gal), N-methylindolyl-beta-D-galactopyranoside, 5-iodo-3-indolyl-beta-D-galactopyranoside, 5-bromo-6-chloro-3-indolyl-beta-D-galactopyranoside, 6-chloro-3-indoxyl-beta-D-galactopyranoside, para-naphtholbenzein-beta-D-galactopyranoside, cyclohexenoesculetin-beta-D-galactopyranoside and 8-hydroxyquinoline-beta-D-galactopyranoside.

3. The method of claim 2, wherein the beta-D-galactopyranoside is 5-bromo-4-chloro-3-indoxyl-beta-D-galactopyranosid (x-Gal).

4. The method of claim 1, wherein the glycosidic bond of the beta D-galactopyranosidse is hydrolyzed by a polypeptide with beta-galactosidase activity.

5. The method of claim 4, wherein the polypeptide with beta-galactosidase activity is the beta-galactosidase from Escherichia coli according to SEQ ID NO: 1 or a variant or an active fragment thereof.

6. The method of claim 1, wherein the cells are obtained from a swab.

7. The method of claim 6, wherein the swab is a swab of the oral mucosa.

8. The method of claim 1, wherein the target DNA sequence to be detected is a bacterial DNA sequence.

9. The method of claim 8, wherein the bacterial DNA sequence is a bacterial resistance gene.

10. The method of claim 9, wherein the bacterial resistance gene is the mecA gene.

11. The method of claim 1, wherein the target DNA sequence to be detected is a viral DNA sequence.

12. The method of claim 11, wherein the viral DNA sequence is a sequence of a human papillomavirus.

13. The method of claim 12, wherein the viral DNA sequence is a sequence of human papillomavirus 16 or 18.

14. The method of claim 1, wherein steps (b) to (d) are carried out successively in one reaction batch.

15. An in vitro method for the detection of a target DNA sequence from a group of DNA sequences, whose members differ from one another in exactly one pre-determined nucleotide position, comprising
(a) providing cells on a support, wherein at least a portion of the cells is lysed;
(b) contacting the cells on the support with first and a second oligonucleotide, at least one of which is labeled with a beta-D-galactopyranoside, which on hydrolysis of the glycosidic bond forms a water-insoluble dye, and wherein said oligonucleotides are selected so that
(i) in each case they in can hybridize by a terminal segment to the DNA target sequence in such a way that the terminal nucleotides of the two terminal segments are arranged immediately adjacent to one another, wherein the nucleotide at the predetermined position of the target DNA sequence undergoes complementary base pairing with a nucleotide in the terminal segment of the first oligonucleotide, and so that
(ii) on hybridization of the two terminal segments to another DNA sequence from the group of DNA sequences due to the absence of complementary base pairing between the nucleotide at the predetermined position of the DNA sequence and a nucleotide in the terminal segment of the first oligonucleotide, the terminal nucleotide of the terminal segment of the first oligonucleotide cannot hybridize to the DNA sequence, so that the terminal nucleotides of the two terminal segments are not arranged immediately adjacent to one another;
(c) incubating the two oligonucleotides under conditions that make hybridization of the two oligonucleotides to the DNA target sequence possible;
(d) contacting the two hybridized oligonucleotides with a ligating agent in contact brings, which only links them to form a ligation product if the two terminal nucleotides of the terminal segments are arranged immediately adjacent to one another;
(e) heating the reaction batch to a temperature at which labeled, nonligated oligonucleotides detach from the target DNA sequence, and at which the ligation product remains hybridized to the target DNA sequence;
(f) detecting the ligation product hybridized to the target DNA sequence by hydrolysis of the glycosidic bond of the beta-D-galactopyranosids;
wherein the formation of a dye in the region of the cells on the support indicates the presence of the target DNA sequence in the cells.

16. The method as claimed in claim 15, wherein the beta-D-galactopyranoside is selected from the group consisting of from 5-bromo-4-chloro-3-indoxyl-beta-D-galactopyranosid (x-Gal), N-methylindolyl-beta-D-galactopyranoside, 5-iodo-3-indolyl-beta-D-galactopyranoside, 5-bromo-6-chloro-3-indolyl-beta-D-galactopyranoside, 6-chloro-3-indoxyl-beta-D-galactopyranoside, para-naphtholbenzein-beta-D-galactopyranoside, cyclohexenoesculetin-beta-D-galactopyranoside and 8-hydroxyquinoline-beta-D-galactopyranoside.

17. The method of claim 16, wherein the beta-D-galactopyranoside is 5-bromo-4-chloro-3-indoxyl-beta-D-galactopyranosid (x-Gal).

18. The method of claim 15, wherein the glycosidic bond of the beta D-galactopyranosidse is hydrolyzed by a polypeptide with beta-galactosidase activity

19. The method of claim 18, wherein the polypeptide with beta-galactosidase activity is the beta-galactosidase from Escherichia coli according to SEQ ID NO: 1 or a variant or an active fragment thereof.

20. The method of claim 15, wherein the target DNA sequence is included in the wild-type or variant sequence of a gene.

21. The method of claim 20, wherein the target DNA sequence is included in the gene coding for the synaptic vesicle protein 2A, which has the sequence shown in SEQ ID NO: 3, or in the variant thereof.

22. The method of claim 21, wherein the oligonucleotides have the sequences shown in SEQ ID NO: 4 and SEQ ID NO: 5.

23. The method of claim 15, wherein the cells are obtained from a swab.

24. The method of claim 23, wherein the swab is a swab of the oral mucosa.

25. The use of an oligonucleotide labeled with a beta-D-galactopyranoside, which on hydrolysis of the glycosidic bond forms a water-insoluble dye, in DNA hybridization methods.

26. The use of claim 25, wherein the beta-D-galactopyranoside is 5-bromo-4-chloro-3-indoxyl-beta-D-galactopyranoside (X-Gal).

27. A kit for carrying out a method of claim 1 or claim 15, which contains at least an oligonucleotide that is labeled with a beta-D-galactopyranoside, which on hydrolysis of the glycosidic bond forms an water-insoluble dye.

28. The kit of claim 27, wherein the beta-D-galactopyranoside is 5-bromo-4-chloro-3-indoxyl-beta-D-galactopyranoside (X-Gal).
